(19) United States
(12) Patent Application Publication
(10) Pub. No.: US 2023/0340597 A1
(43) Pub. Date: Oct. 26, 2023

(54) BIOMARKER PANELS, SYSTEMS, AND METHODS FOR RISK STRATIFICATION OF A SUBJECT FOR ALZHEIMER'S DISEASE

(71) Applicant: URVOGELBIO PRIVATE LIMITED, Hyderabad, Telangana (IN)

(72) Inventors: Sasidhar Venkata MANDA, Hyderabad (IN); Sai Krishnaveni CHEVOORU, Hyderabad (IN); Subhashani PRABHAKAR, Hyderabad (IN); Anusha TUMMALA, Hyderabad (IN)

(21) Appl. No.: 17/912,091

(22) PCT Filed: Mar. 19, 2021
(86) PCT No.: PCT/IN2021/050289

§ 371 Date: Sep. 16, 2022

(30) Foreign Application Priority Data
Mar. 19, 2020 (IN) 201941037878

(51) Int. Cl. C12Q 1/6883

(52) U.S. Cl. CPC C12Q 1/6883; C12Q 2600/158; C12Q 2600/118; C12Q 2600/16

(57) ABSTRACT

The present invention provides biomarker panels, systems, kits, and methods for in vitro diagnosis, prognosis, and risk stratification of AD, based on multi-analyte biomarker profiling. The invention is inexpensive, non-invasive, and does not require the involvement of highly trained personnel. Further, the invention is rapid and provides accurate and objective determination regarding AD's diagnosis and/or prognosis. The invention even enables the early diagnosis of Alzheimer's disease and can facilitate quality lifestyle in AD patients. The invention can also be used for real-time assessment of the outcome of clinical trials and helps develop companion diagnostics for different AD pathways.

### CROSS REFERENCE TO RELATED APPLICATION

This application claims the benefit of priority to Indian Provisional Patent Application Number 201941037878, filed on Mar. 19, 2020, the entire contents of which are hereby incorporated by reference.

### FIELD OF INVENTION

The present disclosure pertains to the field of clinical molecular biotechnology. In particular, the present disclosure relates to methods of prognosis, diagnosis, risk stratification, and therapy monitoring for Dementia and related conditions, including Alzheimer's Disease (AD).

The developed innovation stratifies Dementia patients into the Alzheimer continuum and differentiates AD from other dementias and neurodegenerative diseases. The present disclosure arrives at the stratification with a matrix of 8 protein biomarkers and is complemented with four other clinical parameter groups, including genetic, functional, clinical, and RNA biomarkers. The developed innovation also helps in the development of companion diagnostics for different AD pathways in development.

### BACKGROUND

Alzheimer's disease (AD) is one of the most frequent and important Central Nervous System (CNS) complications affecting memory and cognition. AD is a progressive, degenerative and irreversible disease that slowly destroys memory and thinking skills and eventually leads to the inability to carry on even simple tasks. AD is the most common cause of dementia (a continuous decline in thinking, behavioural and social skills that disrupts a person's ability to function independently). Alzheimer's disease affects almost 47 million worldwide, about 5.8 million people in the United States (The Alzheimer's Association, USA). In India, it affects about 4 million people.

AD progresses in three broad stages, i.e., preclinical, mild cognitive impairment (MCI), and clinical dementia. It is a recognized fact that early diagnosis of Alzheimer's disease leads to better recovery in patients by altering the trajectory of AD, enables access to AD-disease modifying therapies, antioxidant therapies, and other approaches. However, due to lack of early diagnostics, limited diagnostic resources, poverty, inadequate manpower, poor health literacy, most AD cases are detected only at the late clinical dementia stage. Unfortunately, by then, more than 50% of the neurons are already damaged. This, coupled with the lack of an effective cure in place, poses a significant challenge for clinicians and patients. Most of the current drugs are hypothesized to work better with an early on-time diagnosis.

Early diagnosis and/or prognosis of Alzheimer's disease is challenging due to the lack of validated biomarkers that can be used to determine disease status or progression. On average, it takes about 7-32 months for a definitive diagnosis based on clinical, cognition, and medical history testing. The current gold standard for diagnosing AD clinically is limited to establishing a “probable’ or ‘possible’ AD depending on the level of clinical expertise. Symptom-based AD diagnosis is problematic due to significant overlap with other Dementias. The ATN(Xn) Amyloid, Tau tangles, neurodegeneration framework can revolutionize the entire AD diagnosis and distinguish even the early AD, including prodromal and mild cognitive impairment. However, the ability to include biomarker diagnosis currently is restricted to Cerebrospinal fluid tap, which is invasive, expensive, and severely limited to high resource clinical setting. This lack of diagnostic and prognostic methods limits the clinical practitioner in utilizing effective therapeutic strategies.

Currently, AD is detected by quantifying the amyloid load (a signature molecule for diagnosing AD) through amyloid PET. However, this is an invasive, radioactive, expensive, and time-consuming procedure. Further, this procedure is currently restricted to a single place in Mumbai, India. This is still in commission. Further literature has shown that even 30% of healthy individuals have amyloid deposits, resulting in false negatives. The current gold standard for AD diagnosis includes amyloid PET imaging, psychological monitoring, and post-mortem diagnosis by brain biopsy, using immune-histochemical studies to provide diagnostic information. With the current diagnosis system, a conservative estimate estimates that only 10% are diagnosed. An early diagnosis is a viable option where comorbidities and risks favouring AD can be kept in control. Still, the current diagnostic options are not suitable for early diagnosis nor biomarker-based analysis.

Altogether, the currently employed methods exhibit one or more of the following limitations:
They are invasive, which is painful and time taking for the patient.
They are not cost-effective and very elaborate.
They are not commercially available in most parts of the world.
They require trained personnel for carrying out the biopsy and immunohistochemical studies.
Real-time therapy and disease monitoring are not available.
Accurate patient stratification is not possible with the current diagnostic modalities.
Treatment outcomes in clinical trials cannot be immediately assessed in real-time, resulting in delayed therapeutic intervention.

Thus, there is a substantial unmet clinical need in diagnosis, prognosis, and/or treatment of AD. It is extremely important to develop these modalities that can successfully overcome the limitations of the prior art to screen AD to prevent and/or treat the same. The present invention overcomes the prior art problems by solving this long-standing problem for a rapid, non-invasive, and cost-effective determination of Alzheimer's disease. The invention would enable Alzheimer's disease prognosis and diagnosis more accessible and affordable to a vast proportion of the population living around the world.

### SUMMARY OF THE INVENTION

The present invention provides biomarker panels, systems, kits, and methods for in vitro diagnosis, prognosis, and risk stratification of AD, based on multi-analyte biomarker profiling. The invention is inexpensive, non-invasive, and does not require the involvement of highly trained personnel. Further, the invention is rapid and provides accurate and objective determination regarding AD's diagnosis and/or prognosis. The invention even enables the early diagnosis of Alzheimer's disease and can facilitate quality lifestyle in AD patients. The invention can also be used for real-time assessment of the outcome of clinical trials and helps develop companion diagnostics for different AD pathways.

Accordingly, the present disclosure relates to a method of measuring brain biomarker expression (RNA and protein) using serum neuronal exosomes, comprising eight biomarkers, by assaying a biological sample (serum-neuronal exosomes) with ELISAs and PCR. The biological sample obtained is from a subject showing Alzheimer's Dementia. The Eight biomarkers are Abeta 40, Abeta 42 (representing amyloid pathway (A)); Total Tau, phospho Tau 181 (representing Tau tangles (T)); sTrem2 and Neurofilament light (NfL) (representing neurodegeneration (ND)); Neurogranin and REST (representing neuroprotection pathway (NP).

A matrix of amyloid, tau, neurodegeneration, and neuroprotection pathway markers provided an analysis of the staging of Alzheimer's disease. The matrix distinguishes between AD and healthy controls and other neurodegenerative diseases and other dementias, including Vascular Dementia, Frontotemporal dementia, Lewis body, etc. A−T−ND−NP+ are regarded as healthy individuals; A−T−ND−NP− are considered to be prodromal AD; A+T+ND−NP− are regarded as MCI; A+T+ND+NP− are regarded as Alzheimer disease; A+T−ND+NP− are regarded as mixed Dementias; A−T−ND+NP− can be considered as Non-AD, CBD—corticobasal degeneration, FTD—Frontotemporal Dementia; A−T+ND−NP− can be considered as CVD—cerebrovascular disease (CVD), prion and early tauopathy's; A−T+ND+NP− can be considered as Vascular dementia and prion disease.

The sensitivity of the above multi-analyte biomarker analysis is increased with the inclusion of ApoE4 genotyping. Homozygous and heterozygous ApoE4 positive is considered a high-risk for Dementia patients with an increased progression, and ApoE2 genotype is regarded as protective genotype, and ApoE3 genotype is a neutral phenotype. Upregulation of serum HCHO and downregulation of serum ACE-2 activity is associated with AD phenotype. Further immunological changes in blood CBP profile, including a decrease of monocytes and lymphocytes and increased neutrophils, neutrophil to lymphocyte ratio, and platelet to lymphocyte ratio, are associated with AD phenotype.

Upregulation of amyloid, tau and neurodegeneration markers and downregulation of neuroprotection biomarkers are hallmarks of AD dementia. Neuronal damage leads to synaptic dysfunction at the pre and postsynaptic level, which is evident by a decrease of synaptic protein levels (Synaptogamin-1, Synaptopodin, Synaptophysin, VAMP-1, VAMP-2, and GAP-43). Neurological symptoms are characterized by psychological scores, including MMSE, ACE-R, and CDR rating. There is a significant correlation between exosomal biomarkers and psychological scores.

### BRIEF DESCRIPTION OF FIGURES

FIG. 1: Exosomes are released from virtually all cell types. Exosomes released by brain cells can cross the BBB and can be detected in the bloodstream. All cell types in the brain (Neuronal, astrocytes, microglia, oligodendrocytes, and endothelial cells) releases exosomes. Cell-specific exosomes can be enriched from blood samples using exosomal membrane markers and used to detect various proteins and nucleic acids.

FIG. 2: (A) Flowchart and (B) Graphical representation of Neuronal exosomes isolation using CD171 immunoprecipitation method.

FIG. 3: (A) and (B) Flowchart showing isolation of Brain-derived exosomes is from total exosomes. The graph shows (C) Total protein, (D) Lipid, and (E) Acetylcholine esterase estimation in Astrocytes derived exosomes(ADE), Microglial derived exosomes (MDE), and Neuronal derived exosomes (NDE). RNA quality of Brain-derived exosomes (F) ADE, (G) MDE, and (H) NDE was checked using Bioanalyzer.

FIG. 4: Represents the neuron-derived exosomes (NDE) characterization. Figure A represents Transmission electron microscopy (TEM) image of NDEs, Figure B represents DLS analysis represented in number weight Gaussian distribution; Figure C represents Bioanalyzer data for Total exosomes, and neuron derived exosomes, Figure D immunoblotting assay was performed to check the presence of Flotilin and ANXA5 in Neuronal exosomes. Total exosomes (TE) were loaded as the positive control. Figure E represents total protein analysis in NDEs. Figure F represents Total lipid analysis in NDEs. Figure G represents protein/lipid ratio in NDEs, and Figure H represents Acetylcholine esterase (Achei) activity in NDEs Data are expressed as Mean±SD. Scale bar represented in 100 nm for TEM image. Scale bar represented in 100 nm for TEM image. The significance is represented by * where *=p value<0.05, ****pvalue<0.0001.

FIG. 5: Estimation of genetic risk using ApoE genotyping in AD patients compared to healthy control. Figure A represents genotype frequency of ApoE in Healthy and AD groups, Table B represents Number (Percentages) of Various Genotypes in the Disease and Control Groups in a Study of ApoE Polymorphisms, and Table C represents observed Allele Frequencies Among disease and controls using Hardy Weinberg Equilibrium.

FIG. 6: Quantification of AD diagnosis using the functional assay. The data shows (A) a significant increase in serum formaldehyde expression in AD patients than healthy controls. The bar represents mean±standard deviation. The significance is represented by * where *=p value<0.05, ** p-value<0.005. (B) ROC curve of formaldehyde with AUC −0.69 and p value=0.00013 and (C) 95% confidence interval showing column mean the difference among the different AD groups.

FIG. 7: Quantification of AD diagnosis using the functional ACE assay. The data shows (A) a significant decrease in AD patients' ACE expression compared to healthy and between Healthy and patients with other dementias. The bar represents mean±Standard Deviation. The significance is represented by * where *=p value<0.05, ** p-value<0.005. *** p value<0.001, **** p-value<0.0001. The graph shows (B) ROC curve of ACE and (C) 95% confidence interval with column mean the difference among the different AD groups. The tabular representation of ANOVA summary (D) and Turkey's multiple comparison test shows the difference in formaldehyde test analysis among different AD groups.

FIG. 8: Expression of Peripheral blood markers among the different groups of Alzheimer's disease compared to healthy control. The Graph shows (A) significant increase of MPV level in Other dementia compared to early AD, (B) increased Neutrophil's level, (C) a significant decrease in the level of lymphocytes in AD patients compared to healthy, and (D) a significant decrease in the monocytes level in AD patients compared to healthy control. There was an increase in the ratio of (E) NLR and (F) PLR in AD patients compared to healthy control. The bar represents mean±Standard Deviation. The significance is represented by * where *=p value<0.05, ** p-value<0.005. *** p value<0.001, **** p-value<0.0001

FIG. 9: Expression of Neuropathological markers among the different groups of Alzheimer's disease compared to healthy control. The Graph shows (A) significant decrease of Abeta 40 level in AD patients compared to Control, (B) increased Abeta42 level compared to healthy, (C) a significant increase in the level of total tau in AD patients compared to healthy, (D) a significant increase in the phospho 181 tau level in early AD and AD patients compared to healthy control. The graphs show the ROC curve for (E) Abeta 40, (F) Abeta 42, (G) Total tau, and (H) Phospho tau. The bar represents mean±Standard Deviation. The significance is represented by * where *=p value<0.05, ** p-value<0.005. *** p value<0.001, **** p-value<0.0001

FIG. 10: Expression of Neuropathological markers among the different groups of Alzheimer's disease compared to healthy control. The Graph shows (A) significant increase of sTREM2 level in AD patients compared to Control, (B) increased NEFL level in AD patients compared to healthy, (C) a significant decrease in the level of Neurogranin in AD patients and other dementia compared to healthy, (Da) a significant decrease in the REST level in AD patients compared to healthy control. The graph shows the ROC curve for (E) sTREM2, (F) NEFL, (G) Neurogranin, and (H) REST. The bar represents mean±Standard Deviation. The significance is represented by * where *=p value<0.05, ** p-value<0.005. *** p value<0.001, **** p-value<0.0001.

FIG. 11: Dementia screening diagnostic panel.

FIG. 12: Evaluation of Synaptic profile among the different groups of Alzheimer's disease compared to healthy control. The Graph shows (A) significant decrease of Synaptogamin-1 level in AD patients compared to Control, (B) decreased Synaptopodin level in AD patients compared to healthy, (C) decrease in the level of Synaptophysin in AD patients compared to healthy and significant in other dementia compared to early and AD patients. The graphs show the ROC curve for (D) Synaptogamin-1 (SYT), (E) Synaptopodin (SYNPO), and (F) Synaptophysin (SYP). The bar represents mean±Standard Deviation. The significance is represented by * where *=p value<0.05, p-value<0.005. *** p value<0.001, **** p-value<0.0001

FIG. 13: Expression of Synaptic profile among the different groups of Alzheimer's disease compared to healthy control. The Graph shows (A) significant decrease of VAMP2 level in early AD, other dementia and AD patients compared to Control, (B) decreased VAMP1 level in AD patients and other dementia compared to healthy, (C) a significant decrease in the level of GAP43 in AD patients, early AD and other dementias compared to healthy. ROC curve has been shown for (D) VAMP2, (E) VAMP1, and (F) GAP43. The bar represents mean±Standard Deviation. The significance is represented by * where *=p value<0.05, ** p-value<0.005. *** p value<0.001, **** p-value<0.0001.

FIG. 14: Association between Addenbrooke's score (ACE) and (A) Amyloid markers and (B) Synaptic markers in Alzheimer's patients. Pearson Correlation was performed using GraphPad Prism version 9. The expression of biomarkers is represented by blue-red color. Gene Expression decreases from Blue to red color. Descriptive statistics analysis showing a correlation between Addenbrooke's score (ACE) and (C) Amyloid markers and (D) Synaptic markers in Alzheimer's patients.

FIG. 15: Association between Minimental State Examination (MMSE) and (A) Amyloid markers and with (B) Synaptic markers in Alzheimer's patients. Pearson Correlation was performed using GraphPad Prism version 9. The expression of biomarkers is represented by blue-red color. Gene Expression decreases from Blue to red color. Descriptive statistics analysis showing a correlation between MMSE and (C) Amyloid markers and (D) Synaptic markers in Alzheimer's patients.

FIG. 16: Association between Disease Duration (years) and (A) Amyloid markers and with (B) Synaptic markers in Alzheimer's patients. Pearson Correlation was performed using GraphPad Prism version 9. The expression of biomarkers is represented by blue-red color. Gene Expression decreases from Blue to red color. Descriptive statistics analysis showing a correlation between Disease Duration (years) and (C) Amyloid markers and (D) Synaptic markers in Alzheimer's patients.

FIG. 17: Association between Clinical Dementia Rating (CDR) and (A) Amyloid markers and with (B) Synaptic markers in Alzheimer's patients. Pearson Correlation was performed using GraphPad Prism version 9. The expression of biomarkers is represented by blue-red color. Gene Expression decreases from Blue to red color. Descriptive statistics analysis showing a correlation between Clinical Dementia Rating (CDR) and (C) Amyloid markers and (D) Synaptic markers in Alzheimer's patients.

FIG. 18 represents the bar graph showing a comprehensive ranking of housekeeping genes according to employed algorithms for stability assessment FIG. 19: Evaluation of Molecular Diagnosis among the different groups of Alzheimer's disease using Multiplexing PCR profiling. The Graph shows (A) a significant decrease of APP level in AD patients compared to Control, (B) decreased MAPT level in AD patients and other dementia compared to healthy, (C) a significant decrease in the level of NEFL in AD patients compared to healthy. ROC curve is shown for (D) APP, (E) MAPT, and (F) NEFL. The bar represents mean±Standard Deviation. The significance is represented by * where *=p value<0.05, ** p-value<0.005. *** p value<0.001, **** p-value<0.0001.

FIG. 20: Procedure for isolation of neuronal exosome from plasma

FIG. 21: Assay system for determining Alzheimer's disease risk in a subject

### DETAILED DESCRIPTION OF THE INVENTION

The present invention discloses biomarker panels, assay systems, methods, kits for prognosis, diagnosis, in vitro diagnosis, and risk stratification for Alzheimer's disease.

For the first time, the inventors have developed biomarker panels, assay systems, and methods for in vitro diagnosis, prognosis, and risk stratification based on several AD biomarkers' expression levels.

Based on the expression levels of biomarkers derived from biological fluids or samples, the invention enables determination and risk stratification for AD. The invention also enables minimally invasive and real-time determination of the therapeutic modality outcomes adopted for patients. The invention is also helpful in determining the outcome of a new AD drug during clinical trials.

The present invention represents an advancement over the existing methods for the determination of AD. The following features characterize the advances:
(a) Affordable: The diagnostic, prognostic, and risk stratification methods developed in the present invention are inexpensive compared to existing diagnostic or prognostic methods as they are minimally invasive and do not require the involvement of trained personnel and instruments.
(b) Sensitive and Specific: The measurement of multiple biomarker levels enables accurate and objective diagnosis and/or prognosis of Alzheimer's disease and stages of AD and the ability to distinguish from other Dementias.
(c) Rapid: The test results can be ascertained within a short period and associated with less pain.
(d) Risk stratification: The present invention methods would enable accurate characterization of an individual for risk of developing AD.
(e) Companion diagnostics: The present invention methods would enable the development of companion diagnostics for AD patient identification and monitor new drugs.

Thus, the technical problem to be solved in this invention is the rapid, inexpensive, and minimally invasive diagnosis and/or prognosis of Alzheimer's disease and differentiate it from other dementias and healthy counterparts.

The inventors have solved the above problem by developing biomarker panels, assay systems, kits, and methods for in vitro diagnosis, prognosis, and risk stratification based on multi-analyte biomarker profiling using biological fluids or samples.

Unless defined otherwise, all technical and scientific terms used herein have the same meaning as commonly understood by one of ordinary skill in the art to which the methods belong. Although any method, device, kit, reagent, or composition similar or equivalent to those described herein can also be used in the practice or testing of the methods, representative illustrative methods and compositions are now described.

It is appreciated that certain features of the methods, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as an antecedent basis for the use of such exclusive terminology as “solely,” “only,” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other embodiments without departing from the scope or spirit of the present methods. Any recited method can be carried out in the order of events recited or in any other order that is logically possible.

In the present invention, the term “exosomes” refers to small vesicles having a membrane structure secreted from various cell types. Exosomes have diameters of about 30 to 150 nm, and fusion between plasma membranes and multivesicular bodies occurs, thereby being released outside the cells. These exosomes are then purified, and subpopulations of brain exosomes are isolated for multianalyte biomarker analysis

The term “risk stratification,” according to the invention, allows for the classification of patients based on the severity of the AD and, optionally, determining the fatality risk.

The term “biomarker” or “marker” or “genetic marker” refers to any gene, protein, or metabolite whose level of expression in a tissue, cell, or bodily fluid is dysregulated compared to that of a normal or healthy cell, tissue, or biological fluid. Biomarkers to be measured in the invention methods are selectively altered when a subject has developed or is at risk of developing AD.

The term “biomarker panel,” “biomarker profile,” or “biomarker fingerprint” refers to a set of biomarkers. As used herein, these terms can also refer to any form of the biomarker that is measured. Thus, if NEFL is part of a biomarker panel, then either NEFL mRNA, for example, or protein, for example, could be considered to be part of the panel. While individual biomarkers are useful as diagnostics, combination of biomarkers can sometimes provide greater value in determining a particular status than single biomarkers alone. Specifically, the detection of a plurality of biomarkers in a sample can increase the sensitivity and/or specificity of the test.

The term “assay system” defines means for detecting a desired activity which is encapsulated within a screening unit. An assay system directly or indirectly produces a detectable and/or measurable signal when it contacts or reacts with a biomarker or biomarker panel of the present invention. The assay system as used herein is a PCR-based assay system or an immunoassay system.

The term “disease risk” refers to an individual (for example, a human) with a predisposition to suffer from a certain disease, such as AD. This predisposition can be genetic (for example, a specific genetic tendency to suffer the disease, such as hereditary disorders) or obey other factors (for example, age, weight, environmental conditions, exposure to harmful compounds present). in the environment, etc.

The term “in vitro method” refers to a biochemical process performed in a test tube or other laboratory instrument. The term implies that said method is carried out in a biological sample isolated from the subject from whom it is taken.

The term “diagnosis,” as used herein, refers to the determination of a condition of a subject, such as determining whether the subject has a particularly disease condition, susceptibility, or other trait.

The term “prognosis” in the present disclosure relates to predicting disease progression without considering any reference to therapy/treatment/drug. Quantitative or qualitative way of prognosis includes age, genetic risks, expression levels of the various neuropathological biomarker, psychometric testing, neuroimaging, etc. Prognosis is done to prognosis relates to understanding the long-term outcome of a disease.

Before the biomarker panels, assay systems, methods of diagnosis/prognosis, kits comprising reagents for detection of the biomarkers, and other embodiments of the present disclosure are disclosed and described; it is to be understood that the terminologies used herein are to describe particular embodiments only and are not intended to be limiting. It must be noted that, as used in the specification and the appended claims, the singular forms “a,” “an,” and “the” include plural referents unless the context dictates otherwise.

This invention is based on an approach to identify expression levels of multiple biomarkers expressed in AD and develop in vitro and non-invasive systems, methods, and kits for AD diagnosis and/or prognosis. This approach contrasts with earlier approaches in which a single or a small number of biomarkers are quantified to determine AD, which might not be conclusive.

The present invention also allows the categorization of patients into different risk categories for AD and able to identify subtypes of Dementias.

### Development of Alzheimer's Biomarker Panel

In one embodiment, the invention provides for developing a biomarker panel for AD (Alzheimer's Disease/Biomarker Panel), which can also distinguish various Dementia subgroups.

In another embodiment, the Alzheimer's Biomarker Panel contains at least one biomarker specific to the disease.

The AD Panel biomarkers may be protein biomarkers, peptide biomarkers, or nucleic acid biomarkers in another embodiment.

In some embodiments of the methods and kits of the invention, the AD Panel includes at least one biomarker selected from the following non-limiting list: Aβ40, Aβ42, APP, BACE1, TT, MAPT, p-tau181, NfL, FABPs, SYT, SYP, GAP43, SYNPO, VAMP1, VAMP2, PD1, PDL1, PDL2, CTLA4 and APOE genotyping.

A representative list of the biomarkers used in the AD Panel is provided in Table 1.

TABLE 1
Biomarkers in Alzheimer's Biomarker Panel

| | Description of | | | | |
|---|---|---|---|---|---|
| S. No | Marker/Gene | Symbol | Pathway/Factor | Accession No | Gene ID |
| 1 | Amyloid β 40 | Aβ40/Abeta | Amyloid Pathway | NC_000021.9 | 351 |
| | | 40 (protein) | | | |
| | | APP (gene) | | | |
| 2 | Amyloid β 42 | Aβ42/Abeta | Amyloid Pathway | NC_000021.9 | 351 |
| | | 42 (protein) | | | |
| | | APP (gene) | | | |
| 3 | Microtubule- | MAPT (gene)/ | Tau Pathway | NC_000017.11 | 4137 |
| | associated | Total tau | | | |
| | protein Tau | (protein) | | | |
| 4 | Phosphorylated | p-tau181/ | Tau Pathway | NC_000017.11 | 4137 |
| | Tau 181 | Phospho 181 | | | |
| | | Tau | | | |
| 5 | Soluble triggering | sTREM2 | Neuroinflammation | NC_000006.12 | 54209 |
| | receptor expressed | | | | |
| | on myeloid cells 2 | | | | |
| 6 | Neurogranin | NRGN | Neuroprotection | NC_000011.10 | 4900 |
| 7 | RE-1 silencing | REST | Neuroprotection | NC_000004.12 | 5978 |
| | transcription faactor | | | | |
| 8 | Neurofilament | NEFL | Axonal | NC_000008.11 | 4747 |
| | light chain | | degeneration | | |
| 9 | Fatty-acid-binding | FABPs | Axonal | NC_000001.11 | 2170 |
| | proteins | | degeneration | | |
| 10 | Synaptotagamin 1 | SYT 1 | Synaptic profile | NC_000012.12 | 6857 |
| 11 | Synaptophysin | SYP | Synaptic profile | NC_000023.11 | 6855 |
| 12 | Growth associated | GAP43 | Synaptic profile | NC_000003.12 | 2596 |
| | protein 43 | | | | |
| 13 | Synaptopodin | SYNPO | Synaptic profile | NC_000005.10 | 11346 |
| 14 | Vesicle associated | VAMP1 | Synaptic profile | NC_000012.12 | 6843 |
| | membrane protein 1 | | | | |
| 15 | Vesicle associated | VAMP2 | Synaptic profile | NC_000017.11 | 6844 |
| | membrane protein 2 | | | | |
| 16 | Programmed cell death | PD1/CD279 | Immune checkpoint | NC_000002.12 | 5133 |
| | protein 1/cluster of | | inhibitors | | |
| | differentiation 279 | | | | |
| 17 | Programmed death- | PDL1/CD274/ | Immune checkpoint | NC_000009.12 | 29126 |
| | ligand 1/cluster of | B7-H1 | inhibitors | | |
| | differentiation 274/ | | | | |
| | B7 homolog | | | | |
| 18 | Programmed death- | PDL2 | Immune checkpoint | NC_000009.12 | 80380 |
| | ligand 2 | | inhibitors | | |
| 19 | Cluster of | CTLA4/CD152 | Immune checkpoint | NC_000002.12 | 1493 |
| | differentiation | | inhibitors | | |
| | 152 | | | | |
| 20 | Cathepsin D | CTSD | Other factors | NC_000011.10 | 1509 |
| 21 | Visinin like | VSNL | Other factors | NC_000002.12 | 7447 |
| | protein 1 | | | | |
| 22 | Neurofilament | NEFH | Axonal | NC_000022.11 | 4744 |
| | heavy | | degeneration | | |
| 23 | Prostaglandin- | COX2 | Inflammation | NC_000001.11 | 5743 |
| | endoperoxide synthase | | | | |
| | 2/cyclooxygenase-2 | | | | |
| 24 | Placental growth | PGF | Neuroprotection | NC_000014.9 | 5228 |
| | factor | | | | |
| 25 | Apolipoprotein E | APOE | Genetic risk | NC_000019.10 | 348 |

In another embodiment, the biomarker panel for determining Alzheimer's disease risk in a subject comprising Abeta 40, Abeta 42, Total Tau, Phospho 181 Tau, sTREM2, N EFL, Neurogranin, and REST as core markers.

In another embodiment, the biomarker panel optionally at least one biomarker selected from a group comprising Synaptogamin-1, Synaptopodin, Synaptophysin, Vamp-2, VAMP-1, GAP 43, CTSD, VSNL, PD1, PDL2, CTLA4, PDL1, FABP, NEFH, COX2, and PGF.

In another embodiment, the AD Panel may be supplemented with results from one or more diagnostic tests such as, but not limited to psychological scoring, Mini-Mental State Examination (MMSE), Addenbrooke examination, C Magnetic resonance imaging (MRI), and/or Positron emission tomography (PET) scan to obtain the disease diagnosis in this disclosure.

TABLE 2
Clinical Parameters and sub-parameters
used for the development of AD Panel

| Parameter | Sub parameter |
|---|---|
| Genetic risk | ApoE genotyping |
| Neuropathological | Amyloid pathway |
| biomarkers | Tau pathway |
| | Neurodegeneration pathway |
| | Neuroprotection |
| | Synaptic dysfunction |
| Functional assays | Angiotensin Enzyme levels |
| | HCHO assay |
| Psychometric testing | Minimental state examination (MMSE) |
| | Addenbrooke Examination (ACE) |
| | Clinical Dementia Rating (CDR) |
| Neuroimaging | MRI |
| | PET |

### Collection of Biological Samples from a Subject

The biological samples or fluids collected for this invention are collected through non-invasive/minimally invasive means. The biological samples used in this invention are different from traditionally used samples, such as surgical or biopsy samples post-death for confirmatory analysis.

In one embodiment, a biological sample is collected in a non-invasive manner.

In another embodiment, the biological sample is salivary.

In yet another embodiment, the biological sample is a blood or serum sample.

In yet another embodiment, the biological sample is a urine sample.

In yet another embodiment, the biological sample is a tear sample.

In yet another embodiment, the biological sample is a sweat sample.

In yet another embodiment, the biological sample is a cerebrospinal fluid (CSF) sample.

In another embodiment, the biological sample can be harvested using standard techniques known in the art.

This is a case-control, blinded study. Patients were recruited from Apollo hospital, Neurology division, Nizams Institute of Medical science (NIMS), Neurology division with informed consent. This study was approved by the Apollo hospital ethical committee and the Nizams Institute of Medical science (NIMS) ethical committee.

Age and gender-matched healthy controls were recruited. Addenbrooke's cognitive examination (ACE R), Clinical Dementia Rating Scale (CDR), and Mini-mental State Examination (MMSE) analysis were documented in the study.

8 ml of whole blood was collected in EDTA coated tubes, incubated for 10 min at room temperature. PBMCs and plasma were isolated using lympoprep (STEMCELL) at 800 g for 30 min at room temperature. Collected plasma was stored in 0.5 ml aliquots at −80° C. DNA, RNA, and protein were isolated from PBMCs using nucleon spin TRIPEP kit (Macherey-Nagel).

6 ml of whole blood was collected in plain vacutainer, incubated at room temperature for 30 minutes, and centrifuged for 15 mins at 2500 rpm. The resulting serum was collected and stored in 0.5 ml aliquots at −80° C.

### Isolation of Exosomes or Microvesicles

In a further embodiment, the exosomes or microvesicles are isolated from the collected biological samples.

In another embodiment, exosomes or microvesicles specific to one or more cells present in the brain are isolated.

In another embodiment, the isolated exosomes or microvesicles are specific to one or more brain cell types, including neurons, astrocytes, oligodendrocytes, microglia, etc brain endothelial cells, or brain epithelial cells.

In another embodiment, the exosomes or microvesicles are isolated using one or more antibodies.

In another embodiment, the antibodies used for isolating exosomes or microvesicles are specific to one or more proteins/markers selected from L1CAM, MAPT, GLAST, GFAP, OMV, MOG, MBP, CD13, Claudin-5, CD31, GLUT1, and other related neuronal subtypes.

In another embodiment, one or more antibodies used for isolating exosomes or microvesicles are selected from a group comprising anti-L1CAM antibodies, anti-MAPT antibodies, anti-GLAST antibodies, anti-GFAP antibodies, anti-OMV antibodies, anti-MOG antibodies, anti-MBP antibodies, anti-CD13 antibodies, anti-Claudin-5 antibodies, anti-CD31 antibodies, anti-GLUT1 antibodies, and anti-SNAP25 antibodies.

In another embodiment, one or more antibodies used for isolating brain-derived or neuronal exosomes or microvesicles are selected from a group comprising anti-L1CAM antibodies, anti-MAPT anti-SNAP25 antibodies.

In another embodiment, one or more antibodies used for isolating astrocyte-derived exosomes or microvesicles are selected from a group comprising anti-GLAST antibodies and anti-GFAP antibodies.

In another embodiment, one or more antibodies used for isolating oligodendrocyte-derived exosomes or microvesicles are selected from a group comprising anti-OMV antibodies, anti-MOG anti-MBP antibodies.

In another embodiment, one or more antibodies used for isolating microglia-derived exosomes or microvesicles are selected from a group comprising anti-CD13 antibodies.

In another embodiment, one or more antibodies used for isolating brain endothelial cell-derived exosomes or microvesicles are selected from a group comprising anti-Claudin-5 antibodies, anti-CD31 antibodies, and anti-GLUT1 antibodies.

The techniques for the separation of exosomes or microvesicles are known to a person skilled in the art.

In one embodiment, the exosomes or microvesicles can be isolated from any biological sample using standard techniques known, as will be clear to those of skill in the art.

In another embodiment, the exosomes or microvesicles are isolated from the biological samples, including one or more techniques, including, but not limited to PEG precipitation, size exclusion chromatography, usage of protamine chloride, differential centrifugation with PEG/Dextran, and the like.

### Isolation of Neuronal Exosome from Plasma

In brief, 500 ul of plasma was incubated with 15 ul of thromboplastin-D (Thermo Fisher Scientific, MA) for 30 min, and 0.485 ml of Dulbecco's phosphate-buffered saline (DPBS, Thermo Fisher Scientific) with protease and phosphatase inhibitor cocktails (Thermo Fisher Scientific) was added and centrifuged at 4500×g for 20 min at 4 C.

The supernatants were collected, then 150 ul of Total exosome isolation reagent for plasma was added and incubated at room temperature for 10 min. After centrifugation at 10,000 g×g for 5 min at room temperature, pellets were resuspended in sterile water with protease and phosphatase inhibitor cocktails and incubated overnight in a rotating mixer (4° C.). Then 4 ug of anti-human CD171 antibody (bioscience), Thermosphere Scientific) and 50 ul of 3% BSA (SRL, INDIA) were added and incubated for 1 hour in a rotating mixer at 4° C. Then, 15 μl of streptavidin plus ultra-link resin (Thermo Fisher Scientific) containing 25 μl of 3% BSA was added. After centrifugation at 200×g for 10 min at 4° C., the pellet was resuspended in 200 μl 0.1 M glycine-HCl (pH=3.0) vortexing for 30 seconds.

This is followed by centrifugation of samples for 5 min at 4500 g. The supernatants were collected, and to this, 15 ul TRIS HCL was added. This suspension contains intact EVs, so an aliquot was used for exosomal characterization. 200 ul of MPER and 25 ul of 3% BSA were added to the remaining rest (intact EVs) and subjected to 2 freeze-thaw cycles.

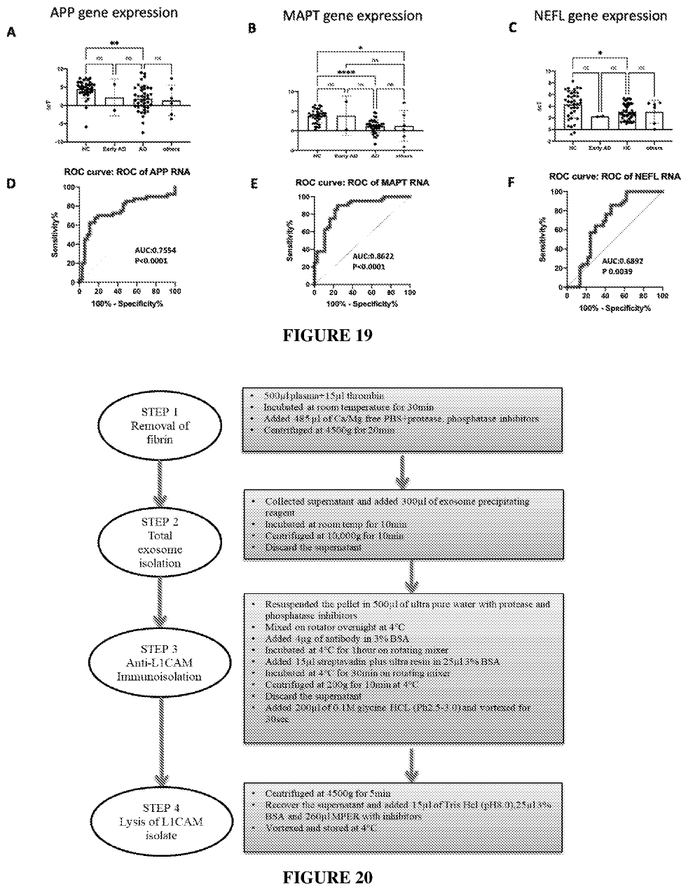

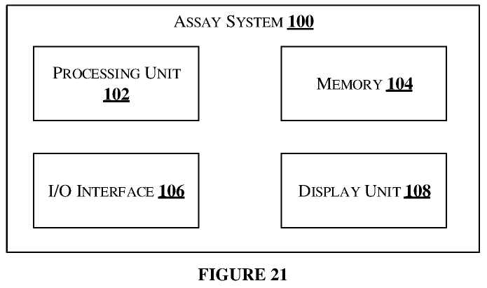

The procedure for isolation is depicted in FIG. 20.

### Checking Purity, Potency, and Quality of Isolated Exosomes/Microvesicles

In another embodiment, one or more biochemical assays, bio-analyzation studies, biophysical characterization, and molecular biological characterization are performed to check the quantity and quality of the isolated exosomes or microvesicles.

In another embodiment, the total protein content of the isolated exosomes or microvesicles is checked to assess the exosomes or microvesicles' quality. Total protein content may be checked using any suitable technique, as will be clear to those of skill in the art.

In another embodiment, the isolated exosomes or microvesicles' lipid content is measured to assess the quality of the exosomes or microvesicles. Lipid content may be checked using any suitable technique, as will be clear to those of skill in the art.

In another embodiment, the lipid to protein ratio of the isolated exosomes or microvesicles is measured to assess the exosomes or microvesicles' quality. Lipid to protein ratio may be measured using any suitable technique, as will be clear to those of skill in the art.

In another embodiment, the Acetyl CoA esterase content of the isolated exosomes or microvesicles is measured to assess the isolated exosomes or microvesicles' quality. Acetyl CoA content may be checked using any suitable technique, as will be clear to those of skill in the art.

In another embodiment, the Dipeptidyl peptidase IV (DPPIV) content of the isolated exosomes or microvesicles is measured to assess the exosomes or microvesicles' quality. DPPIV content may be checked using any suitable technique, as will be clear to those of skill in the art.

In another embodiment, the isolated exosomes or microvesicles' nucleic acid content is measured to assess the quality of the exosomes or microvesicles. Nucleic acid content may be checked using any suitable technique, as will be clear to those of skill in the art.

In another embodiment, the size of the isolated exosomes or microvesicles is checked to assess the exosomes or microvesicles' quality. The size may be checked using any suitable technique, as will be clear to those of skill in the art. Exemplary embodiments of techniques used for determining the size of the exosomes include, but are not limited to, dynamic light scattering (DLS) microscopy, transmission electron microscopy (TEM), and the like.

In another embodiment, the charge on the exosomes or microvesicles is checked to assess the exosomes or microvesicles' quality. The charge may be checked using any suitable technique, as will be clear to those of skill in the art. Exemplary embodiments of charge determination include, but is not limited to, zeta potential.

In another embodiment, the nucleic acid cargo of the exosomes or microvesicles is checked to assess the exosomes or microvesicles' quality. The nucleic acid cargo may be determined using any suitable technique, as will be clear to those of skill in the art. Exemplary embodiments of nucleic acid cargo determination include, but is not limited to, determination by polymerase chain reaction (PCR), Next-Generation Sequencing (NGS), and the like.

### Neuronal Exosome Characterization

### Total Protein Analysis from NDEs

Total intact NDEs protein contents were measured using a BCA protein assay kit (Thermo Scientific Pierce, Rockford, Ill., USA). BSA standard or samples (25 μl) were transferred to a 96 well plate to which 200 μl working reagent was added (working reagent 50:1 ratio of assay reagents A and B). The plate was incubated for 30 min at 37° C. and analyzed at 562 nm using an ELISA reader (Biotek H synergy).

### Estimation of Acetylcholine Esterase Activity from NDEs

Acetylcholine esterase activity assay was used to detect NDEs' presence in our isolates, as it has been previously established that AChE is enriched in exosomes (2). The procedure was done as described previously by Savina et al. and Febbario et al. Briefly to 2 ul of the NDEs, 198 ul of working reagent [0.1M phosphate buffer, 1.25 mM Acetylcholine iodide, 0.1 mM 5,5′-dithiobis (2-nitrobenzoic acid)] was added. Yellow colour developed is read in ELISA reader at 412 nm. The enzyme activity was expressed as U/mg of protein.

### Total Lipid Analysis

The total lipid content of NDEs was analyzed using the protocol described earlier (5). 1 mg/ml of 1,2-Dioleoyl-sn-glycerol-3-phosphocholine (DOPC) was used as lipid standard. Briefly, 200 ul of 96% sulphuric acid was added to 40 ul of lipid standards or total NDEs. After brief vortexing, the open test tubes were incubated at 90° C. in a fume hood for 20 min. The tubes were then cooled down to RT by placing at 4° C. for 5 min, and 120 μL of phospho-vanillin reagent was added to each tube and vortexed. Then, 280 μL of each sample was transferred to a 96 well plate and incubated for one hr at 37° C. The color developed was read with an ELISA reader at 540 nm.

The protein/Lipid ratio was calculated to check the integrity or quality of exosomes.

### DLS Analysis

DLS was used to assess the size of the particles present in the exosome isolation fraction. The exosomal fraction was briefly diluted to 1:1000 with 1× PBS in a 1 mL (1 cm×1 cm) plastic cuvette at 23° C. The size dispersion was measured using Nicomp nano Z3000 dynamic light scattering (DLS).

### Transmission Electron Microscopy (TEM)

TEM was performed at the Center for Cellular and Molecular Biology, Hyderabad. Freshly isolated NDEs were fixed using 2.5% Glutaraldehyde in PBS buffer at pH7.2 for 1 hour at 4° C.

Then the exosomes were postfixed in 1% osmium tetroxide for 1 hour at 4 C. Next, the samples were dehydrated for 10 min in increasing concentrations of alcohol. The samples were embedded in an embedding mixture and polymerized at 65 C for 24 hours. Then, sections were prepared using an ultra-thin microtome and then were stained with 2% uranyl acetate for 10 min and with lead acetate for 10 min at room temperature (6). Finally, the grid was observed under JEM-2100 plus Transmission electron microscopy (JOEL) at 120 kV.

### Immunoblotting Analysis

Western blot analysis was carried on total exosomes and neuronal exosomes from plasma.

The proteins were separated on 12% SDS-Polyacrylamide Gel, and the separated protein were transferred on 0.2 μm PVDF membrane.

After the transfer, the blot was blocked with 3% BSA in 1× TBST for 1 hr on a shaker. The membrane was washed 3 washes with TBST for 5 minutes each. Primary antibody tagged against Flotillin-1 (49 kDa) and ANEXA5 (58 kDa) were added to the blot and incubated overnight at 4° C.

The blot was washed thrice with TBST for 10 min each and incubated with HRP-conjugated secondary antibody (1:5000 dilution) for 1 hr at room temperature. The membrane was washed thrice with TBST for 5 minutes intervals and visualized under chemiluminescence.

### Bioanalyzer

### RNA Isolation from Total and Neuron Derived Exosomes

Total RNA was extracted by using TRIZOL LS reagent (Thermo fisher scientific) according to the manufacture's protocol. Briefly, to 250 ul of full EVs and NDEs, 750 ul of Trizol Ls was added, vortexed, and incubated at room temperature for 10 min. Then, 200 ul of chloroform was added, incubated at room temperature for 10 min, and centrifuged at 12000 g for 15 min.

After centrifugation, the upper aqueous layer was separated, to that isopropanol, glycoblue co-precipitant (Thermo fisher scientific), and tRNA was added and incubated overnight in −20 C. The RNA was isolated by centrifuging at 12000 g for 15 min at 4° C. The supernatant was discarded carefully, and the pellet was washed twice with 70% ethanol at 10000 g for 10 min at 4° C. The supernatant was discarded carefully with a pipette, and the RNA pellet was air-dried for 10 minutes.

The RNA dissolved in DEPC treated water. The RNA concentration, purity, and integrity from total EVs and NDEs were determined by Agilent 2100 Bioanalyzer with RNA 6000 pico kit (Agilent Technologies, CA, USA). The RNA was converted to cDNA using an iScript cDNA synthesis kit (Bio-Rad Laboratories, Inc, US). 1 μg total RNA was mixed with 4 μl of 5× iScript Reaction Mix and 1 μl of iScript Reverse Transcriptase and made up to 20 μl with RNase free water. The sample was briefly centrifuged and amplified at 25° C. for 5 min, 46° C. for 20 min, and 1 min at 95° C. The cDNA synthesized was kept at −20° C.

### Measuring the Expression Levels of Alzheimer's Biomarker Panel mRNA Transcript in Isolated Exosomes or Microvesicles

RNA is isolated from exosomes as described above for the bioanalyzer analysis. Further steps include primer design and real-time PCR. In another embodiment, the expression levels of one or more genetic markers as listed in Table 1 are measured using multiplex polymerase chain reaction (PCR), multiplex enzyme-linked immunosorbent assay (ELISA), and a wide variety of functional or biochemical assays.

In one embodiment, the expression level is determined at the nucleic acid level. Typically, the expression level of a gene may be determined by assessing the quantity of mRNA. Methods for assessing the quantity of mRNA are well known in the art.

In some embodiments, mRNA or total RNA is isolated from the exosomes of the biological samples and quantified through polymerase chain reactions (PCR).

In some embodiments, the methods for determining the expression level of mRNA comprise the steps of providing total RNAs extracted from and subjecting the RNAs to amplification and hybridization to specific probes, more particularly by means of a quantitative or semi-quantitative RT-PCR.

In one embodiment, the expression level is determined at the protein level. Typically, the expression level of genetic markers at the protein level may be determined by ELISA. Methods for ELISA, such as a sandwich, competitive, or non-competitive ELISA or immunoassay formats, are well known in the art.

### Primer Design

The primers were designed using NCBI primer design software.

### List of Gene of Interest

| | | | Base |
|---|---|---|---|
| Genes | Forward 5′-3′ | Reverse 3′-5′ | pair |
| APP | CCATCAGGGA | CAGTTCAGGG | 75 |
| | CCAAAACCTG | TAGACTTCTT | |
| | C | GGC | |
| | (SEQ ID NO: 1) | (SEQ ID NO: 2) | |
| MAPT | CCAGCTCTGC | GCGATCCCCT | 89 |
| | GACTAAGCAA | GATTTTGGAG | |
| | (SEQ ID NO: 3) | G | |
| | | (SEQ ID NO: 4) | |
| CTSD | ACAGGCACTT | GGGGATCATG | 99 |
| | CCCTCATGGT | TACTCGCCCT | |
| | (SEQ ID NO: 5) | (SEQ ID NO: 6) | |
| VSNL | CGGCTTAAGC | GCTTCCCCAT | 110 |
| | GTTTACCCGA | CCTGCAGTTA | |
| | (SEQ ID NO: 7) | ((SEQ ID NO:8) | |
| PD1 | CGTGGCCTAT | ATCCCTTGTC | 106 |
| | CCACTCCTCA | CCAGCCACTC | |
| | (SEQ ID NO: 9) | (SEQ ID NO: 10) | |
| PDL2 | CAGTGCTATC | GCCAGGTGTT | 182 |
| | TGAACCTGTG | GGCTAGTCTT | |
| | GT | (SEQ ID NO: 12) | |
| | (SEQ ID NO: 11) | | |
| CTLA4 | CATGATGGGG | TCAGTCCTTG | 92 |
| | AATGAGTTGA | GATAGTGAGG | |
| | CC | TTC | |
| | (SEQ ID NO: 13) | (SEQ ID NO:14) | |
| PDL1 | AAATGGAACC | GATGAGCCCC | 104 |
| | TGGCGAAAGC | TCAGGCATTT | |
| | (SEQ ID NO: 15) | (SEQ ID NO: 16) | |
| FABP | TGGGCACCTG | AGCAAAACCC | 71 |
| | GAAGCTAGTG | ACACCGAGT | |
| | (SEQ ID NO: 17) | (SEQ ID NO: 18) | |
| NEFH | CGAGGAGTGG | GCGCATAGCG | 70 |
| | TTCCGAGTGA | TCTGTGTTCA | |
| | G | (SEQ ID | |
| | (SEQ ID NO: 19) | NO: 20) | |
| STMN2 | TTCAGCAAGA | GCCTCTCATT | 191 |
| | TGGCGGAGGA | GCTTCTCTCC | |
| | (SEQ ID NO: 21) | TT | |
| | | (SEQ ID NO: 22) | |
| COX2 | GGAGAAAACT | GTGCACTGTG | 75 |
| | GCTCAACACC | TTTGGAGTGG | |
| | GGA | G | |
| | (SEQ ID NO: 23) | (SEQ ID NO: 24) | |
| NEFL | GCTATGCAGG | TCACGTTGAG | 100 |
| | ACACGATCAA | GAGGTCTTGG | |
| | CA | T | |
| | (SEQ ID NO: 25) | (SEQ ID NO: 26) | |
| PGF | CTCGTCAGAG | CAGGGAGACA | 139 |
| | GTGGAAGTGG | CAGGATGGGC | |
| | TA | (SEQ ID NO: 28) | |
| | (SEQ ID NO: 27) | | |

### List of Housekeeping Genes

| | | | Base |
|---|---|---|---|
| Genes | Forward 5′-3′ | Reverse 3′-5′ | pair |
| L13/ | TCCTTTCCGC | GGGCCTTACG | 168 |
| RPL13 | TCGGCTGTTT | TCTGCGGAT | |
| | (SEQ ID NO: | (SEQ ID NO: | |
| | 29) | 30) | |
| 18s | CTGAGAAACG | GCCTCGAAAG | 107 |
| | GCTACCACAT | AGTCCTGTAT | |
| | C | TG | |
| | (SEQ ID NO: | (SEQ ID NO: | |
| | 31) | 32) | |
| Bactin | GTCTGCCTTG | TCGAGGACGC | 103 |
| | GTAGTGGATA | CCTATCATGG | |
| | ATG | (SEQ ID NO: | |
| | (SEQ ID | 34) | |
| | NO: 33) | | |
| GAPDH | CCACTAGGCG | GACCAAATCC | 95 |
| | CTCACTGTTC | GTTGACTCCG | |
| | T | AC | |
| | (SEQ ID NO: | (SEQ ID NO: | |
| | 35) | 36) | |
| B2M | CCTGCCGTGT | GCTGCTTACA | 70 |
| | GAACCATGTG | TGTCTCGATC | |
| | A | CCA | |
| | (SEQ ID NO: | (SEQ ID NO: | |
| | 37) | 38) | |
| PBGB/ | CAGCCTACTT | CCTGTGGTGG | 202 |
| HMBS | TCCAAGCGGA | ACATAGCAAT | |
| | (SEQ ID NO: | GA | |
| | 39) | (SEQ ID NO: | |
| | | 40) | |

### Real-Time PCR

To study the expression of genes specific to AD studies, Total RNA was isolated from the early AD, AD, other dementia, and healthy control tissue exosomes for quantitative Real-Time PCR (Applied Biosystems). For the amplification, 0.5 μl of cDNA was added to 2× SYBR Green master mix (Takara), 200 nM Forward, and Reverse primer, and volume was adjusted using nuclease-free water. The components of the master mix were briefly spined before setting the reaction. Negative control with nuclease-free water instead of cDNA was added for detecting any contamination.

The pre-amplification reaction included initial denaturation at 95° C. for 2 mins, followed by 20 cycles of 95° C. for 5 seconds, 60° C. for 30 seconds and 72° C. for 1 min. Then Real-time PCR amplification was continued on the pre-amplified product, which included initial denaturation at 95° C. for 2 mins, followed by 20 cycles of 95° C. for 5 seconds, 60° C. for 30 seconds, and final melt curve analysis using SYBR green dye. The primers were checked for sensitivity and specificity. Primer efficiency and LOD were detected before using the Primers for the amplification. The Gene of interest and housekeeping genes used for quantification are listed above.

### Quantifying ApoE Genotyping and Distribution of ApoE Genotype among Dementia Patients and Specifically Alzheimer Disease

Genomic DNA isolation from PBMCs: According to the manufacturer's instructions, genetic DNA was extracted from PBMCs by NucleoSpin® tripep mini kit for RNA, DNA, and protein purification.

The purity of the DNA extracted was assessed using the Eppendorf nanodrop. 260/280 ratio was used to determine protein contamination, while 260/230 was used to determine guanidine contamination.

ApoE genotyping assay was done using commercial Taqman SNP assay from Thermofischer in a blinded manner. For rs429358 polymorphism, the assay ID used is C_3084793_20. For rs7412 polymorphism assay ID used is C_904973_10. For the TaqMan SNP Genotyping Assay, 5 mL of TaqMan reagent (TaqMan genotyping Master Mix), 0.5 mL of 20× Tag Man SNP Genotyping Assay reagent, 1 ml of genomic DNA (100 ng/ml), and 3.5 mL of sterile water was added.

After a pre-heating step at 95° C. for 20 seconds, 40 reaction cycles were performed using the Applied Biosystems 7500 Real-Time PCR System platform (Applied Biosystems, Foster City, Calif., USA) with a denaturation step at 95° C. for 3 seconds and annealing and extension at 60° C. for 30 seconds each. After the procedure, the genotypes were determined using the TaqMan genotyping software.

### Measuring the Levels of Serum Formaldehyde and Serum ACE-2 Levels among Dementia Patients and Specifically Alzheimer Disease

In another embodiment, serum formaldehyde expression levels of one or more markers specific to Alzheimer's disease are measured. Measurement of the expression level of markers/genes specific to Alzheimer's disease can be performed by various techniques well known in the art.

### Serum ACE-2 Levels: Quantification Includes Three Steps, i.e. Preparation of Plasma Samples, Standard Preparation, and Reaction Preparation

Preparation of plasma samples: 80 μl plasma was mixed with 20 μl 50% Trichloroacetic (TCA). The solution was vortexed and kept on ice for 10 minutes. Then, it was centrifuged at 9100×g for 10 minutes at 4° C. The supernatant was transferred into a fresh centrifuge tube, and the pellet was discarded.
Standard preparation: For preparing the standard, 125 ng (0.12 ul) of stock solution was added to (Sodium borate) NaB buffer to make the total volume 100 ul. It was then serially diluted by adding 50 ul to the next tube containing 50 ul of NaB buffer ,making the concentration 62.5 ng. The standard was diluted till the concentration reached 0.244 ng.
Reaction preparation: In the blank tube, we need to add 12.5 ul of 3.5 mM HHL and 5 ul water. Next, in the standard tube, 12.5 ul 3.5 mM hippuryl-L-histidyl-L-leucine (HHL), 5 ul ACE (Enzyme) was added. In the sample tube, 12.5 ul 3.5 mM HHL and 5 ul Plasma supernatant was added.

The Blank, Standard, and Sample tubes were then incubated at 37° C. for 30 minutes. After the incubation, 75 ul of 0.34M NaOH was added to each tube and mixed thoroughly with the pipette. Next, 10 ul of o-phthaldialdehyde (OPA) was added to each of the mixture and pipetted thoroughly, and incubated at room temperature for 10 minutes. Further, 25 ul of 3M HCL was added to all the tubes and quantified on a fluorescent plate reader at 355 nm excitation; 535 nm emission.

Serum Formaldehyde levels—Formaldehyde assay was done as previously described by Mooto Nakijima et al. 5 ul of the plasma sample solution is mixed with 100 ul of 0.1M phosphate buffer (pH5.5). To that, 10 ul of ethy 3-amino crotonate was added and incubated at 37° C. for 15 min. The reaction mixture's fluorescence is measured at excitation 375 nm, and emission 465 nm, and the concentration of formaldehyde is determined by plotting the standard calibration curve.

### Measuring the Expression Levels of Alzheimer's Biomarker Panel Markers in Isolated Exosomes or Microvesicles

In another embodiment, the expression levels of one or more markers specific to Alzheimer's disease is measured. Measurement of the expression level of markers/genes specific to Alzheimer's disease can be performed by a variety of techniques well known in the art.

The lysed NDE protein levels were used. Human neurofilament protein L (NFL, cusabio), SYP (cusabio), REST (cusabio), GAP43 (ImmunoTag), VAMP1 (ImmunoTag), VAMP2 (ImmunoTag), Synaptopodin (SYNPO) (ImmunoTag), A^{β}1-42 (MyBioSource), Synaptotagmin-1 (SYT-1) (ImmunoTag), pTau (My BioSource), A^{β}1-40 (ImmunoTag), Neurogranin (ImmunoTag), Total Tau (ImmunoTag), sTREM (My BioSource) and human CD81 (cusabio), a tetraspanin exosome marker, were measured with an enzyme-linked immunosorbent assay (ELISA) according to the manufacturer's protocol. The protein levels were normalized to the CD81 protein, pan exosome marker.

In another embodiment, the expression levels of one or more markers selected from Aβ40, Aβ42, TotalTau, p-tau181, NfL, sTrem2, Neurogranin and REST, are measured using ELISA to determine the expression level of the protein for the development of the diagnostic panel.

In another embodiment, the expression levels of one or more markers selected from Synaptogamin-1, Synaptopodin, Synaptophysin, VAMP-1, VAMP-2, and GAP-43 are quantified by ELISA to determine the expression level of the protein for the development of a companion diagnostic for assessing Synaptic dysfunction and loss during AD.

In another embodiment, the mRNA expression levels of one or more markers selected from APP, MAPT, C TSD, VSNL, PD, PDL2, CTLA4, PDL1, FABP, STMN2, COX2, and NfL are measured using PCR to determine the expression level of the gene.

In another embodiment, the mRNA expression levels of housekeeping genes (L13, 18SRNA, GAPDH, β2M PDGM and actin) are measured using PCR to determine the gene's expression level of the ideal housekeeping gene/s for normalization of the gene expression.

### Recording Psychological Scores of the Dementia Patients for Correlation

Psychological scores of the patients were established through three different scales, i.e., Addenbrooke's cognitive examination (ACE), Clinical Dementia Rating (CDR), and Minimental State examination (MMSE)

Addenbrooke's cognitive examination (ACE R): ACE is a brief neuropsychological assessment of cognitive functions. The diagnostic capability of ACE has given it the status of a reliable bedside cognitive tool. The shortcomings of the ACE test were modified in the ACE-R test. The test is widely used for determining mild cognitive impairment and dementia. The test includes measures, language, memory, visuospatial skills, and orientation. ACE-R is easy to use and has excellent sensitivity and diagnostic accuracy.
Clinical Dementia Rating Scale (CDR)—The CDR was developed at Washington University School of Medicine and was first published in 1982. It was last revised in 1993. The CDR has established inter-rater reliability and can be administered by trained personnel (usually a clinician or trained nurse). It employs a semi-structured interview with both the patient and a reliable informant (usually a spouse or adult children) to rate performance in six domains: memory, orientation, judgment, problem-solving, community affairs, home and hobbies, and personal care. Each part is placed (independent of dysfunction caused by non-cognitive factors) according to one of five impairment levels: 0=none, 0.5=questionable, 1=mild, 2=moderate, 3=severe. A CDR of 0 indicates no dementia. CDRs of 0.5, 1, 2, and 3 show very mild, mild, moderate, and severe dementia, respectively.
The Mini-mental state Examination—MMSE was developed as a brief screening instrument for MCI and AD. The MMSE is sensitive for detecting MCI and mild AD in the general population. A score ≤of 24 was found to be the optimal cut-off point for a diagnosis of cognitive impairment. Using the MMSE had a sensitivity of 18% to detect MCI, whereas the MoCA saw 90% of MCI subjects. In the mild AD group, the MMSE had a sensitivity of 78%, whereas the MoCA detected 100%. Specificity was excellent for both MMSE and MoCA (100% and 87%, respectively).
Correlation of the above scores along with disease duration is conducted to observe a biomarker-clinical similarity.

### Statistical Analysis

GraphPad Prism 9.0 software (San Diego, Calif., USA) was used for statistical analysis. Patients are divided into early AD, A,D and other dementia based on neuropsychological assessment in the demographic factors. The student's t-test was used for comparisons involving continuous variables. Fisher's exact test and the student's t-test were used for comparing gender and age distribution. Statistical significance was noted at p<0.05. ROC curve was used to assess the significance of the ELISA data. Clinical correlation was done using Pearson's correlation coefficient. One-way ANOVA was used for descriptive statistics.

### Diagnosis or Prognosis of Alzheimer's Disease

The present invention is based on the fact, that expression levels of some biomarkers differ significantly in subjects affected with Alzheimer's disease and dementia, as opposed to healthy subjects.

In one embodiment, one or more biomarkers used for in diagnosis of Alzheimer's disease is selected from a group comprising APP, MAPT, CTSD, VSNL, PD1, PDL2, CTLA4, PDL1, FABP, STMN2, COX2, and NfL.

Diagnosis or prognosis of any clinical condition contains the following phases: (i) the examination phase, involving the collection of data; (ii) the comparison of these data with standard/reference values (iii) the finding of any significant deviation, i.e., a symptom, during the comparison; and (iv) the attribution of the deviation to a particular clinical picture.

Measurement of the expression levels of one or more biomarkers through neuronal exosome profiling of the biological sample provides a sound basis to find any significant deviation from the standard values. Consequently, it can be determined whether the deviation is due to Alzheimer's disease or any related Dementias in the subject.

### Development of Alzheimer's Biomarker Panel

The etiology of Alzheimer's disease is unknown. Several biomarkers can capture the complex and heterogeneous biology of Alzheimer's disease from Dementia.

In one embodiment, the expressional levels of at least one candidate biomarker for Alzheimer's disease shall be used to develop a biomarker panel.

In another embodiment, the panel is generated based on the data generated during the screening stage, feasibility analysis stage (biomarker prioritization and imaging), and development stage (assay optimization and training).

In another embodiment, several biomarkers in genetic risk biomarkers, neuropathological biomarkers from neuronal exosomes (RNA and protein markers), and functional biomarkers will be integrated to identify the disease and differentiate it from healthy controls.

Receiver operator characteristic (ROC) curves are generated as standard summaries of diagnostic accuracy and disease status. The above biomarkers' discriminatory potential will be verified by the clinical parameters and will be correlated to the above biomarkers.

The biomarker panel can be used for risk stratification and monitoring the prognosis of Alzheimer's disease.

### Neuronal Exosomes as a Potential Diagnostic Marker—Workflow

Exosomes are small nanoscale vesicles of 30-150 nm in diameter, which help cell-cell communication and homeostasis. These exosomes are shed from different host cells and interacts with other cells by releasing their genetic material, protein, and lipid into extracellular space. The most exciting feature of exosomes is that it echoes with its parent cells' characteristics in protein and nucleic acid and altering recipient cells' function. This property of holding the genetic material them an ideal biomarker. Moreover, exosomes can cross the blood-brain barrier (BBB) and enter the peripheral system. All cell types in the brain (Neuronal, astrocytes, microglia, oligodendrocytes, and endothelial cells) releases exosomes. Cell-specific exosomes can be enriched from blood samples using exosomal membrane markers and used to detect various proteins and nucleic acids. This study used neuronal exosomes, a non-invasive platform to characterize the brain-derived biomarkers through blood-derived neuronal exosomes.

### Schematic Representation of the Workflow

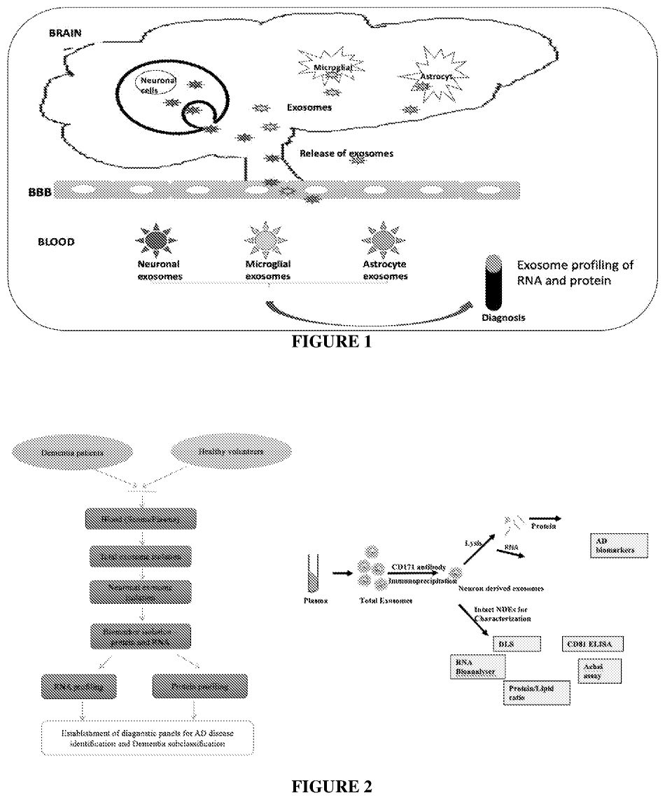

Blood collection was conducted from two cohorts after informed consent—healthy age-matched controls and dementia patients. From plasma, total exosomes were isolated and using cell-specific exosomal marker antibody (neuronal—CD171), neuronal derived exosomes (NDE) were isolated (FIG. 2).

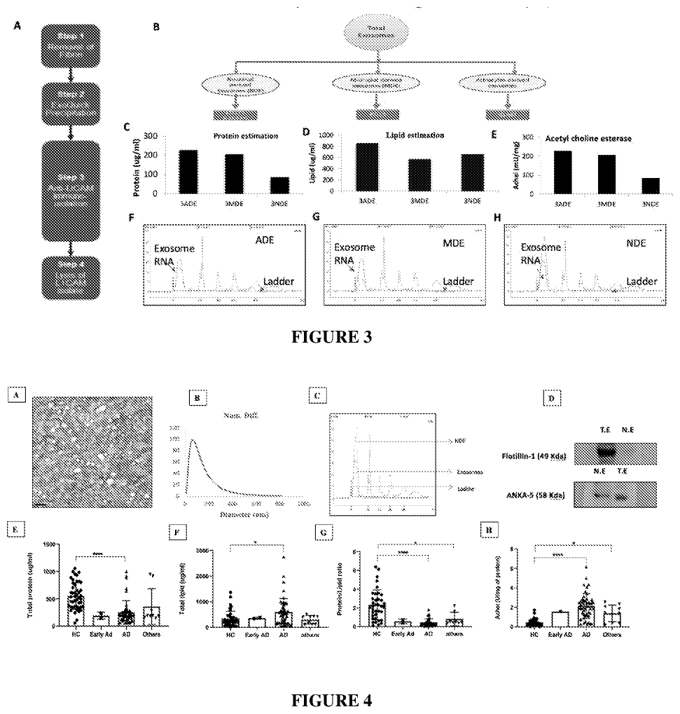

Establishment of brain-derived exosomes (neurons, microglia, and astrocytes) isolation: Total exosomes were isolated from plasma using different methods (8K PEG, 6K PEG, 12kPEG, 4K PEG, PEG DEX, Protamine chloride, kit method). We proceeded further with the Exosome isolation kit (Qiagen). Cell-specific exosomes were isolated from neurons, microglial, and astrocyte cells using marker antibodies (CD171, CD13, and GLAST antibodies). The quality of Neuron-derived exosomes (NDE), Microglial-derived exosomes (MDEs), and astrocyte-derived exosomes (ADE) were confirmed by protein, lipid, and acetyl choline esterase activity. The RNA content of the NDE, MDE, and ADE revealed no ribosomal RNA (18sRNA and 28rRNA) seen in the cell-specific exosomes. The RNA peak for NDE, MDE, and ADE were observed in the range of 25-200 nucleotides (FIG. 3F, G and H). Based on the above data, we confirmed that NDE, MDE, and ADE were successfully isolated from plasma.
Confirmation of neuronal derived exosome isolation: The morphology of NDE isolation was confirmed by Transmission electron microscopy (FIG. 4A). The quality of NDE isolation was confirmed by measuring the size of the particle using Dynamic light scattering (DLS) analysis (NICOMP Nano Z3000) which showed that vesicles are in the range of 87.6±13.0 nm in diameter (FIG. 4B). The NDE RNA content revealed that there is no ribosomal RNA (18sRNA and 28rRNA) seen in the NDEs. The RNA peak for NDEs was observed in the range of 25-200 nucleotide (FIG. 4C). In NDEs, exosomal markers protein expression (Flotillin-1 and ANAX-5) was confirmed using western blotting (FIG. 4D). Furthermore, total protein/lipid ratio and acetylcholine esterase activity were also done, which indirectly assessed the NDEs' quality (FIG. 4E, F, G, and H). Based on the above data, we confirmed that NDEs were successfully isolated from plasma.

### Demographics Characteristics of AD Patients

In total, 51 patients are AD, 2 had early AD, and 10 patients had other dementias. Also, 46 healthy age-matched controls (HC) were included as a control group. The demographic details of the study subjects are included in TABLE 3. The mean age for Alzheimer's patients was 68.40±8.018, whereas, for early Alzheimer's dementia, it was 60.50±6.364, and for other dementias, it was 64.30±9.730, for controls mean age was 62.63±11.96. For Alzheimer's patients, an average number of schooling years was 11.12±4.616; for early Alzheimer's, it was 13±1.414, for other dementia it was 13.75±2.712, for controls, it was 12.85±3.3999. For Alzheimer's patients average mean years of cognitive impairment was 2.757±1.680; for early Alzheimer's, it was 2±0, and for other dementias, it was 2.022±1.468. Mostly the patients had cognitive impairment for 2 to 3 years across all groups. Social factors like religion, social strata were also included in the study. For Alzheimer's patients mean MMSE score was 16.02±4.848; for early AD, it was 21.50±0.7071, and for other dementia, it was 20.67±3.905. For Alzheimer patients, the mean ACE score was 58.51±20.95; for early AD, it was 86±7.071, and for other dementia was 70.11±8.667. For Alzheimer's patients mean CDR score was 1.598±0.8012; for early AD, it was 0.5±0, and for other dementia, it was 1.190±0.7166. Even comorbidities were assessed for controls and diseased patients. Among 46 controls, 14 patients have diabetes, and 10 patients have hypertension; in early AD, 1 patient had hypertension; in Alzheimer's patients, 12 have diabetes and 39 have hypertension and in other dementia 4 have diabetes, and 3 had hypertension.

TABLE 3
Demographical characteristics of AD patients

| | | | | | Dementia | |
|---|---|---|---|---|---|---|
| S. | | | | Early | Alzheimer's | Other |
| No | | | Control | Alzheimer's | Disease | Dementia |
| 1 | Number | | 46 | 2 | 51 | 10 |
| 2 | Gender | | | | | |
| 3 | | Female | 26 | 1 | 28 | 4 |
| 4 | | Male | 20 | 1 | 23 | 6 |
| 5 | Age (years) | | 62.63 | 60.50 | 68.40 | 64.30 |
| | Mean (Std. Dev) | | (11.96) | (6.364) | (8.018) | (9.730) |
| 6 | Number of | | 12.85 | 13.00 | 11.12 | 13.75 |
| | schooling years | | (3.399) | (1.414) | (4.616) | (2.712) |
| | Mean (Std. Dev) | | | | | |
| 7 | Years of cognitive | | | 2.000 | 2.757 | 2.022 |
| | impairment | | | (0.000) | (1.680) | (1.468) |
| | Mean (Std. Dev) | | | | | |
| 7 | Psychological | | | | | |
| | monitoring | | | | | |
| 8 | | MMSE | | 21.50 | 16.02 | 20.67 |
| | | Mean | | (0.7071) | (4.848) | (3.905) |
| | | (Std. Dev) | | | | |
| 9 | | (ACE) | | 86.00 | 58.51 | 70.11 |
| | | Mean | | (7.071) | (20.95) | (8.667) |
| | | (Std. Dev) | | | | |
| | | CDR | | 0.5000 | 1.598 | 1.190 |
| | | Mean | | (0.000) | (0.8012) | (0.7166) |
| | | (Std. Dev) | | | | |
| 10 | Diabetes | | | | | |
| | | Present (n) | 14 | 0 | 12 | 4 |
| | | Absent (n) | 32 | 2 | 39 | 6 |
| 11 | Hypertension | | | | | |
| | | Present (n) | 10 | 1 | 22 | 3 |
| | | Absent (n) | 36 | 1 | 29 | 7 |

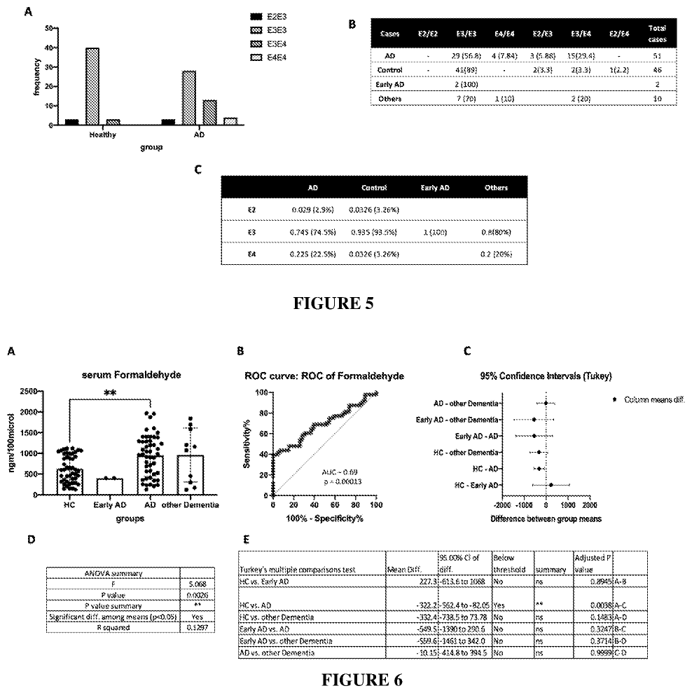

Apo E allele genotyping frequency—In AD we found a high genotype frequency of E3/E3 (56.8%), followed by E3/E4 (29.4), E2/E3 (5.8%) and E4/E4(7.84%). In Early AD, it was E3/E3 only. In other dementia, the genotype frequency was 70% for E3/E3, 10% for E4/E4, and 20% for E3/E4. In controls the genotype frequency was 89% for E3/E3, 3.3% for E2/E3, 3.3% for E3/E4 and 2.2% for E2/E4 (FIG. 5A and B). Allele frequencies of ApoE genotyping were determined using Hardy Weinberg Equilibrium, where we found a high frequency of allele E3 (74.5%) followed by E4 (22.5%) and E2 (2.9%) in the AD group (FIG. 5C). In Early AD, only the E3 allele was seen. In other dementias, the allele frequency for E3 was 80%, and for E2, it was 20% (FIG. 5C). We have observed that AD E4/E4 genotype frequency was high compared to other dementia and controls. The APOE genotyping may help diagnose AD, especially in patients presenting with atypical features or early-onset dementia.

### Establishing AD Diagnosis Using Formaldehyde Test

Formaldehyde is an environmental pollutant generated in substantial amounts in the human body during normal metabolism. This aldehyde is a well-established neurotoxin that affects memory, learning, and behavior. Also, in several pathological conditions, including Alzheimer's disease, an increase in the expression of formaldehyde-generating enzymes and elevated levels of formaldehyde in the brain have been reported. Brain cells have the potential to generate and dispose of formaldehyde. Although moderate formaldehyde concentrations are not acutely toxic for brain cells, repeated exposure to formaldehyde severely affects theft metabolism in astrocytes and neurons. These formaldehyde-induced alterations in brain cells' metabolism may contribute to the impaired cognitive performance observed after formaldehyde exposure and neurodegeneration in diseases associated with increased formaldehyde levels in the brain. In this study, we have analyzed the formaldehyde levels in plasma using fluorometric estimation. Plasma formaldehyde levels were high in AD and other dementia groups when compared to controls. The ROC for formaldehyde assay showed significant AUC of 0.69, p 0.00013) respectively (FIG. 6B). The optimal cut-off index was 1139 ng/100 ul with 39.58% sensitivity and 100% specificity with 27.02% to 53.69% of 95% Confidence intervals (Table 5). The optimal cut-off was derived using the youdens index. From the above results, it is confirmed that estimation of formaldehyde from plasma in AD patients add additional value to AD diagnosis in differentiating from the control group.

### Quantifying of AD Diagnosis Using Functional ACE-2 Activity

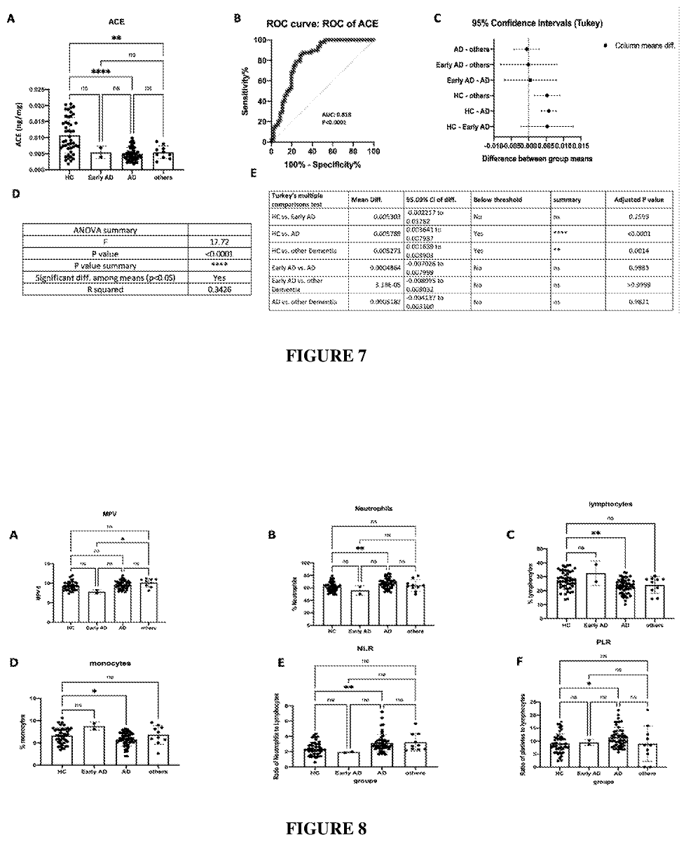

In this study, we have observed a significant decrease in AD (0.005789, p<0.0001) when compared to healthy control group (FIG. 7A). There is a significant decrease in other dementia groups (0.005271; p=0.0014) compared to the healthy control group. No significant difference was observed between the control and early AD group. The ROC curve for the ACE assay showed a significant AUC of 0.818, p<0.0001, respectively (FIG. 7B). The optimal cut off index was 0.007215ng/mg with 87.5% sensitivity and 69.57% specificity with 75.30% to 94.14% of 95% Confidence intervals (Table 5). The optimal cut-off was derived using the youdens index. The above results confirmed that estimation of ACE activity from plasma in AD patients was elevated compared to the control group. Previous studies have shown that ACE has an important role in Aβ degradation, and the administration of ACE inhibitors promoted the accumulation of Aβ. Previous studies confirm the role of Ace in Aβ degradation, and hence low ACE activity can lead to increased Aβ-mediated neuronal damage, plaque accumulation, and risk of AD. So, estimating ACE activity levels from plasma in relation to AD biomarkers might be more clinically useful in diagnosing AD and other dementias.

### Prediction of Alzheimer Risk from Peripheral Blood Markers

TABLE 4
Peripheral Blood markers levels variation among the different groups of Alzheimer's Disease compared
to Healthy control - Neutrophils, Neutrophil to Lymphocyte ratio (NLR), Platelet to lymphocyte ratio (PLR)
are increased. % lymphocytes and % monocytes are decreased in AD in comparison with healthy controls.

| | Blood parameter | | | | |
|---|---|---|---|---|---|
| | HC | Early AD | AD | Others | HC vs. AD |
| | Mean + SD | Mean + SD | Means | Mean + SD | p value |
| Age (years, | 62.63 (11.96) | 60.50 (6.364) | 68.45 (7.946) | 64.30 (9.730) | 0.456 |
| mean + SD) | | | | | |
| WBC Count (10⁹/L) | 7.612 (1.797) | 6.7 (1.273) | 7.983 (1.657) | 7.927 (2.401) | 0.6848 |
| Neutrophils (%) | 62.47 (9.154) | 57.40 (3.111) | 66.40 (7.945) | 68.53 (6.705) | 0.0037 ** |
| Lymphocyte (%) | 26.95 (7.366) | 29.85 (0.2121) | 23.28 (5.231) | 22.88 (5.041) | 0.0016 ** |
| Monocytes (%) | 6.602 (1.811) | 8.750 (0.9192) | 6.229 (1.988) | 5.170 (6.415) | 0.0495 * |
| Esonophils (%) | 3.702 (2.332) | 2 (1.838) | 3.142 (2.20) | 2.524 (2.510) | 0.4851 |
| Basophils (%) | 0.6310 (0.30) | 0.8 (0) | 0.6065 (0.2585) | 1.295 (2.930) | 0.8103 |
| Haemoglobin | 13.2 (1.818) | 13.8 (2.970) | 12.71 (1.636) | 13.22 (1.725) | 0.5294 |
| Haematocrit | 39.81 (5.071) | 41.60 (8.768) | 39.73 (4.801) | 41.09 (4.869) | 0.9806 |
| RBC Count | 4.605 (0.5673) | 4.510 (0.9758) | 4.545 (0.5411) | 4.529 (0.6321) | 0.9816 |
| MCV (fl) | 86.75 (7.582) | 92.50 (0.7071) | 87.75 (5.786) | 88.62 (11.10) | 0.9964 |
| MCH (fl) | 28.73 (3.108) | 30.55 (0.07071) | 33 (32.66) | 30.11 (2.477) | 0.8077 |
| RDW (fl) | 14.62 (1.778) | 13.65 (0.3536) | 14.78 (2.313) | 14.11 (1.619) | 0.9865 |
| Platelet count | 261.3 (70.07) | 283 (36.77) | 248.9 (70.35) | 167.6 (118.7) | 0.9626 |
| (10³/mm³/L) | | | | | |
| MPV (fl) | 9.251 (1.135) | 7.8 (0.5657) | 9.647 (1.046) | 12.63 (7.958) | 0.4324 |
| MLR | 4.308 (1.619) | 3.429 (0.3360) | 3.774 (1.175) | 3.666 (1.238) | 0.1606 |
| NLR | 2.584 (1.063) | 1.923 (0.09057) | 3.093 (1.192) | 3.220 (1.166) | 0.0043** |
| PLR | 9.489 (3.464) | 9.477 (1.164) | 11.32 (4.189) | 8.995 (6.786) | 0.0319* |

Table 4 shows the peripheral blood markers among different group of Alzheimer's disease compared with healthy controls. In this study, we have observed a significant increase in Neutrophils (%) (66.40±7.945, p=0.0037) in the AD group and other dementia (68.53±6.705) when compared with the control group (FIG. 8 B). We have observed a significant decrease in Lymphocyte (%) in AD (23.28±5.231, p=0.0016) in the AD group when compared with the control group (FIG. 8C). We have observed a slight decrease in Monocytes (%) in the AD group (6.229±1.988, p=0.0495) when compared with the healthy control group (FIG. 8D). We have observed a significant increase in NLR in the AD group (3.093±1.192, p=0.0043) compared with the control group (FIG. 8E). We have observed a substantial increase in PLR in the AD group (11.32±4.189, p=0.0319) compared with the control group (FIG. 8F).

Previous studies have shown that many routine peripheral blood markers may be novel inflammatory markers and may be associated with central nervous system diseases' onset or prognosis. Several studies have shown that during cerebral haemorrhage, there is an increase in neutrophils and lymphocyte counts, which is considered a risk factor. Even in acute cerebral infarction, an increase in NLR and MPV was correlated with mortality in acute cerebral infarction patients. We have hypothesized that routine peripheral blood markers in relation to AD biomarkers might be more clinically useful in diagnosing AD and other dementias. Continuous variables are expressed as mean±standard deviation and were compared by variance analysis. Within-group comparisons were determined using one-way repeat measure of analysis of variance (ANOVA) with Tukey post hoc test.

### Expression of Neuropathological Markers Among Different Groups of AD and Healthy Controls

No single marker can capture the heterogeneity of Dementia and particularly AD. The National Institute of Aging and Alzheimer Association introduced a framework restricted to amyloid PET, tau imaging, and MRI findings. We have extended this framework to include analytical biomarkers including amyloid and Tau pathology and enriched with them neurodegeneration, neuroprotection, and synaptic markers to arrive at an accurate diagnosis of AD, AD continuum and able to distinguish AD from other Dementias and neurodegenerative disease.

### Amyloid Pathology Markers

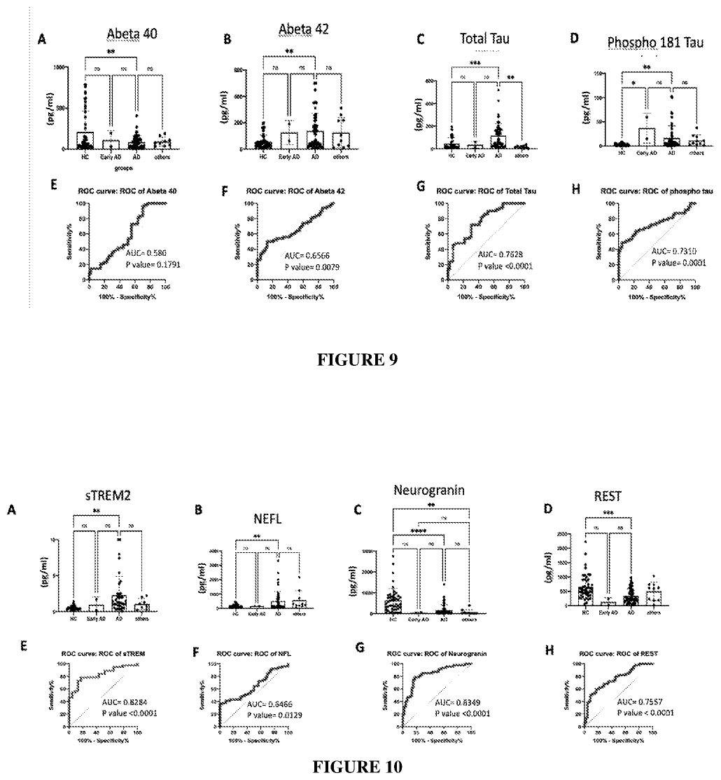

CD81 levels were measured in all samples to normalize the exosomal content. The mean value for all determinations of CD81 in each assay group was set at 1.00, and the relative values for each sample were used to normalize their recovery. Units for all analytes are pg/neuronal-derived exosomes in 1 mL of plasma (pg/ml). We included Abeta 40 and Abeta 42 to represent amyloid pathology. The levels of Abeta40 were significantly downregulated in the AD group compared with healthy controls (FIG. 9A). The ROC for Abeta40 showed an AUC of 0.580, p=0.1791 (FIG. 9E). The optimal cut-off index was 232. pg/ml with 95.83% sensitivity and 95% CI (86.02% to 99.26%) and 29.79% specificity with 18.65% to 43.98% of 95% Confidence intervals (Table 5).The levels of Abeta42 were significantly upregulated in the AD group compared with healthy controls (FIG. 9B). The ROC for Abeta42 showed an AUC of 0.6566, p=0.0079 (FIG. 9F). The optimal cut off index was 111.9 pg/ml with 50% sensitivity and 95% CI (36.64% to 63.36%) and 87.23% specificity with 74.83% to 94.02% of 95% Confidence intervals (Table 5).

### Tau Pathology Markers

We included total tau and phospho tau 181 to represent Tau pathology. The total tau levels were significantly upregulated in the AD group compared with healthy controls (FIG. 9C). The ROC total tau showed an AUC of 0.7628, p<0.0001 (FIG. 9G). The optimal cut off index was 36.35 Pg/ml with 72% sensitivity and 95% CI (58.33% to 82.53%) and 69.57% specificity with 55.19% to 80.92% of 95% Confidence intervals (Table 5). The levels of pTau 181 were significantly upregulated in AD, early AD, and other dementia groups compared with healthy controls (FIG. 9D). The ROC for pTau181 showed an AUC of 0.7310, p=0.0001 (FIG. 9H). The optimal cut off index was 6.78 Pg/ml with 48.94% sensitivity and 95% CI (35.28% to 62.76%) and 95.56% specificity with 85.17% to 99.21% of 95% Confidence intervals (Table 5).

### Neurodegeneration Markers

We included sTrem2 and Neurofilament light (NEFL) to represent neurodegeneration pathology. The levels of sTREM2 were significantly upregulated in AD, early AD, and other dementia groups compared with healthy controls (FIG. 10A). The ROC for sTREM2 showed a significantly higher AUC of 0.8284, p<0.0001 (FIG. 10E). The optimal cut off index was 0.7762 Pg/ml with 78.38% sensitivity and 95% CI (62.80% to 88.61%) and 82.61% specificity with 62.86% to 93.02% of 95% Confidence intervals (Table 5).

### Axonal Degeneration Marker

The levels of NEFL were significantly upregulated in AD and other dementia groups compared with healthy controls (FIG. 10B). The ROC for NEFL showed an AUC of 0.6466, p=0.0129 (FIG. 10F). The optimal cut off index was 452.9 Pg/ml with 35.29% sensitivity and 95% CI (23.63% to 49.01%) and 100% specificity with 92.29% to 100% of 95% Confidence intervals (Table 5).

### Neuroprotection Markers

We included repressor element 1-silencing transcription factor (REST) and Neurogranin to represent neuroprotection pathology. Neurogranin levels were significantly downregulated in AD, early AD, and other dementia groups than healthy controls (FIG. 10C). The ROC for neurogranin showed a significantly higher AUC of 0.8349, p<0.0001 (FIG. 10G). The optimal cut off index was 168.2 Pg/ml with 78.72% sensitivity and 95% CI (65.10% to 88.01%) and 82.61% specificity with 62.86% to 90.91% of 95% Confidence intervals (Table 5). REST levels were significantly downregulated in AD, early AD, and other dementia groups compared with healthy controls (FIG. 10D). The ROC for REST showed a significantly higher AUC of 0.7557, p<0.0001 (FIG. 10H). The optimal cut off index was 310.3 Pg/ml with 60% sensitivity and 95% CI (46.18% to 72.39%) and 82.61% specificity with 69.28% to 90.91% of 95% Confidence intervals (Table 5).

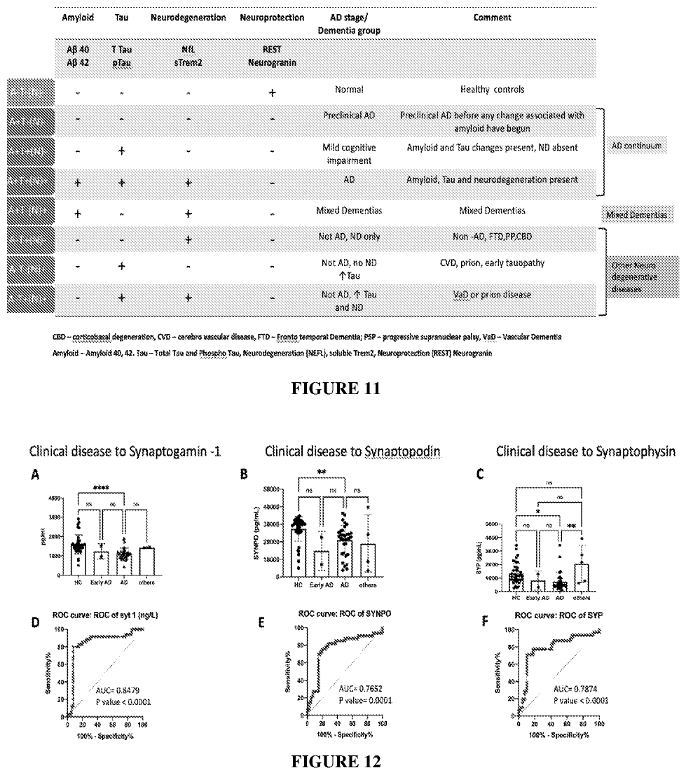

The matrix of neuropathological proteins representing Amyloid, Tau, neurodegeneration, neuroprotection pathway shown above will be used to diagnose the entire AD continuum, differentiate it from other Dementias and other neurodegenerative diseases (FIG. 11)

### Synaptic Profile Markers

The levels of Synaptogamin-1 were significantly downregulated in AD, early AD, and other dementia groups compared with healthy controls (FIG. 12A). The ROC for Synaptogamin-1 showed a significantly higher AUC of 0.8479, p<0.0001 (FIG. 12D). The optimal cut off index was 1226 Pg/ml with 80% sensitivity and 95% CI (64.11% to 89.96%) and 92.5% specificity with 80.14% to 97.42% of 95% Confidence intervals (Table 5). Synaptopodin levels were significantly downregulated in AD, early AD, and other dementia groups compared with healthy controls (FIG. 12B). The ROC for synaptopodin showed a significantly higher AUC of 0.7652, p=0.0001 (FIG. 12E). The optimal cut off index was 25540 Pg/ml with 75.76% sensitivity and 95% CI (58.98% to 87.17%) and 80% specificity with 65.24% to 89.50% of 95% Confidence intervals (Table 5). The Synaptophysin levels were significantly downregulated in AD and early AD groups compared with healthy controls (FIG. 12C). The ROC for Synaptophysin showed a significantly higher AUC of 0.7874, p<0.0001 (FIG. 12F). The optimal cut off index was 624.9 Pg/ml with 70.97% sensitivity and 95% CI (53.41% to 83.90%) and 89.74% specificity with 76.42% to 95.94% of 95% Confidence intervals (Table 5).

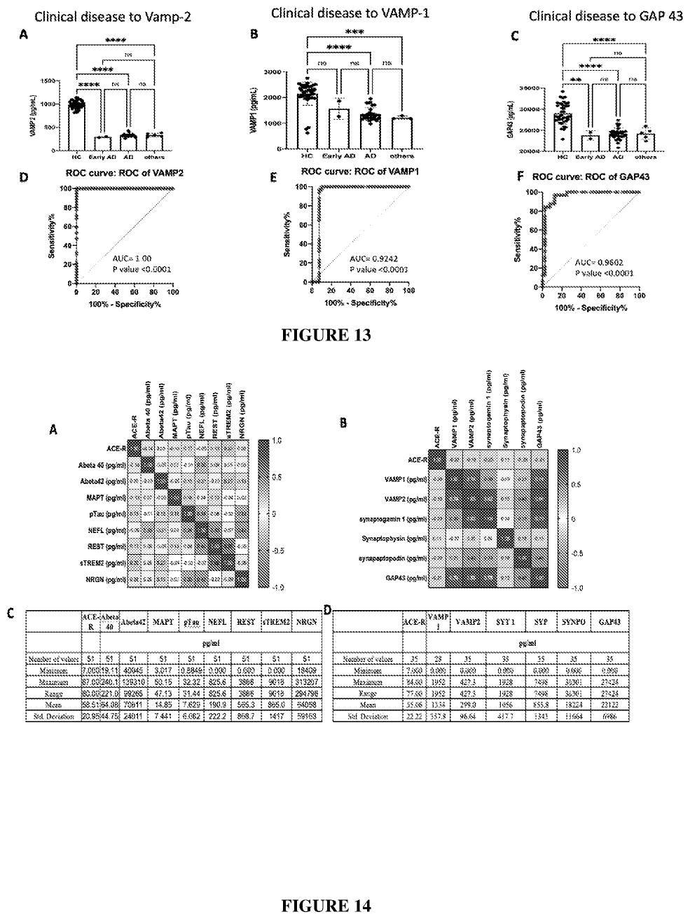

The levels of VAMP2 were significantly downregulated in AD, early AD, and other dementia groups compared with healthy controls (FIG. 13A). The ROC for VAMP2 showed a significantly higher AUC of 1, p<0.0001 (FIG. 13D). The optimal cut off index was 621.1 Pg/ml with 100% sensitivity and 95% CI (89.28% to 100%) and 100% specificity with 91.24% to 100% of 95% Confidence intervals (Table 5). The levels of VAMP 1 were significantly downregulated in AD, early AD, and other dementia groups when compared with healthy controls (FIG. 13B). The ROC for VAMP1 showed a significantly higher AUC of 0.9242, p<0.0001 (FIG. 13E). The optimal cut off index was 1866 Pg/ml with 96.77% sensitivity and 95% CI (83.81% to 99.83%) and 92.5% specificity with 80.14% to 97.421% of 95% Confidence intervals (Table 5). The levels of GAP43 were significantly downregulated in AD, early AD, and other dementia groups compared with healthy controls (FIG. 13C). The ROC for GAP43 showed a significantly higher AUC of 0.9602, p<0.0001 (FIG. 13F). The optimal cut off index was 26552 Pg/ml with 96.88% sensitivity and 95% CI (84.26% to 99.84%) and 87.5% specificity with 73.89% to 94.54% of 95% Confidence intervals (Table 5).

### Correlation of Established Neuropathological Biomarkers to Traditional/Clinical Risk Scores

Dementia involves progressive and often decline in cognition, function, behaviour, and care needs. Traditional assessment in dementia relies on collateral as well as patient-derived information. Three different cognitive scales are traditional used, i.e., Addenbrooke cognitive assessment (ACE-R), mini-mental state examination (MMSE), and clinical dementia rating (CDR). The established biomarkers are correlated to the above scores by Pearson correlation. The correlation coefficient r, ranges from −1 to +1. −1 represents perfect negative correlation; 1 represents perfect positive correlation; −1 to 0, one variable increases as the other decreases; 0. The variable does not vary at all; 0 to 1, the two variables tend to decrease or increase together.

### Correlation of Biomarkers Against Psychometric Testing Addenbrooke's Score (ACE-R)

FIG. 14A shows the heat map of the Pearson correlation matrix between AD biomarkers and ACE-R score. A significant positive correlation was observed between sTREM2 and ACE-R (r=0.20) with a mean value of 865.0 (FIG. 14C), a positive correlation was observed between Abeta42 and ACE-R (r=0.05) with a mean value of 70611, a slight positive correlation was observed between NRGN and ACE-R (r=0.08) with a mean value of 64058, a positive correlation was observed between REST and ACE-R (r=0.13) with a mean value of 565.3, a positive correlation was observed between pTau and ACE-R (r=0.13) with a mean value of 7.629. A negative correlation was observed between Abeta 40 and ACE-R (r=−0.14) with a mean value of 64.08, a negative correlation was observed between MAPT and ACE-R (r=−0.13) with a mean value of 14.85, a negative correlation was observed between NEFL and ACE-R (r=−0.05) with a mean value of 190.9. The above results show that reverse correlation was observed in Abeta 40, MAPT, and NEFL markers compared with ACE-R scores.

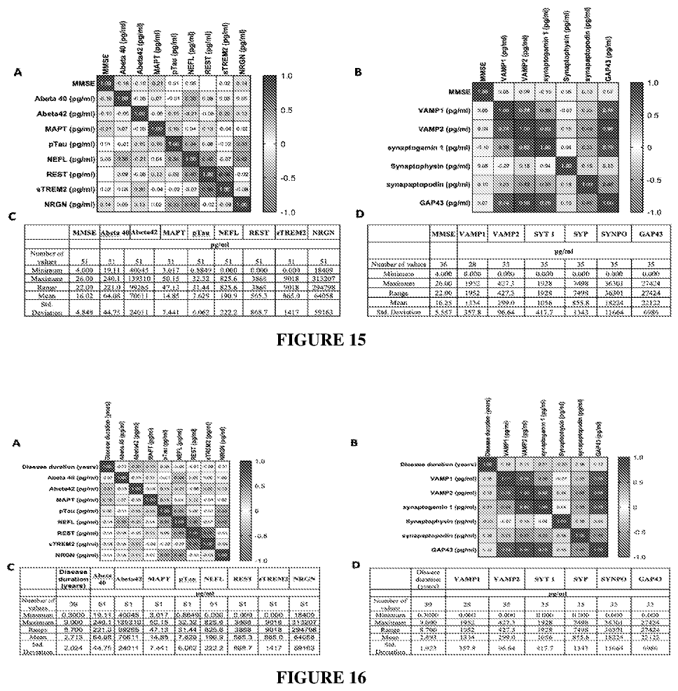

FIG. 14B shows the Pearson correlation matrix's heat map between synaptic profile markers and ACE-R score. A slight positive correlation was observed between synaptophysin and ACE-R (r=0.11) with a mean value of 855.8 (FIG. 15D). A negative correlation was observed between VAMP1 and ACE-R score (r=−0.20) with a mean value of 1334, a negative correlation was observed between VAMP2 and ACE-R (r=−0.18) with a mean value of 299, a high negative correlation was observed between synaptogamin1 and ACE-R (r=−0.23) with a mean value of 1056, a negative correlation was observed between synaptopodin and ACE-R (r=−0.20) with a mean value of 18224A negative correlation was observed between GAP43 and ACE-R (r=−0.21) with a mean value of 22122. The above results show that reverse correlation was observed in VAMP1, VAMP2, synaptogamin1, synaptopodin, and GAP43 markers compared with ACE-R scores.

### Correlation of Biomarkers Against Psychometric Testing Mini-Mental State Examination (MMSE)

FIG. 15A shows the heat map of the Pearson correlation matrix between AD biomarkers and MMSE score. A significant positive correlation was observed between NRGN and MMSE (r=0.14) with a mean value of 64058, a positive correlation was observed between sTREM2 and MMSE score (r=0.02) with a mean value of 865, a positive correlation was observed between NEFL and MMSE score (r=0.05) with a mean value of 190.0a, positive correlation was observed between pTau 181 and MMSE score (r=0.01) with a mean value of 7.629. A significant negative correlation was observed between MAPT and MMSE score (r=−0.21) with a mean value of 14.85, a negative correlation was observed between Abeta42 and MMSE score (r=−0.10) with a mean value of 70611, a positive correlation was observed between Abeta40 and MMSE score (r=−0.10) with a mean value of 16.02. No correlation was observed between REST and MMSE score. From the above results, it is evident that reverse correlation was observed between Abeta40, Abeta42, and MAPT markers, whereas direct correlation was observed between pTau, NEFL, sTREM2 and NRGN and MMSE scores.

FIG. 15B shows the heat map of the Pearson correlation matrix between synaptic profile markers and MMSE score. A significant positive correlation was observed between synaptopodin and MMSE (r=0.10) with a mean value of 18224, a positive correlation was observed between VAMP2 and MMSE (r=0.09) with a mean value of 1334, a positive correlation was observed between GAP43 and MMSE (r=0.07) with a mean value of 22122, a positive correlation was observed between VAMP1 and MMSE score (r=0.05) with a mean value of 16.25a, positive correlation was observed between synaptophysin and MMSE score (r=0.05) with a mean value of 855.5. A negative correlation was observed between synaptogamin1 and MMSE score (r=−0.10) with a mean value of 1056. From the above results, it is evident that reverse correlation was seen only in synaptogamin 1 marker compared with MMSE. In contrast, a direct correlation was observed in VAMP1, VAMP2, synaptophysin, synaptopodin, and GAP43 with MMSE score.

### Correlation of Biomarkers Against Psychometric Testing Disease Duration (Years)

FIG. 16A shows the Pearson correlation matrix's heat matrix between AD biomarkers and disease duration (years). A significant positive correlation was observed between MAPT and disease duration (r=0.31) with a mean value of 14.85, a positive correlation was observed between pate and disease duration (r=0.06) with a mean value of 7.629. A significant negative correlation was observed between Abeta42 and disease duration (r=−0.31) with a mean value of 70611, a negative correlation was observed between Abeta40 and disease duration (r=−0.07) with a mean value of 64.08, a negative correlation was observed between NEFL and disease duration (r=−0.04) with a mean value of 190.9, a negative correlation was observed between REST and disease duration (r=−0.01) with a mean value of 565.3a, negative correlation was observed between sTREM2 and disease duration (r=−0.08) with a mean value of 865.0a, negative correlation was observed between NRGN and Disease duration (r=−0.01) with a mean value of 64058. From the above results it is evident that direct correlation was observed in MAPT and pTau with disease duration. In contrast, reverse correlation was observed in Abeta40, Abeta42, NEFL, REST, sTREM2, and NRGN with disease duration.

FIG. 16B shows the Pearson correlation between synaptic profile markers and disease duration (years). A significant positive correlation was observed between synaptogamin1 and disease duration (r=0.21) with a mean value of 1056, a positive correlation was observed between VAMP1 and disease duration (r=0.16) with a mean value of 1334, a positive correlation was observed between VAMP2 and disease duration (r=0.13) with a mean value of 299, a positive correlation was observed between synaptopodin and disease duration (r=0.16) with a mean value of 18224a, positive correlation was observed between GAP43 and disease duration (r=0.12) with a mean value of 22122. A significant negative correlation was observed between synaptophysin and disease duration (r=−0.20) with a mean value of 855.8. From the above results it is evident that VAMP1, VAMP2, synaptogamin1, synaptopodin, and GAP43 have a direct correlation with disease duration, whereas reverse correlation was observed between synaptophysin and disease duration.

### Correlation of Biomarkers Against Psychometric Testing Against Clinical Dementia Rating (CDR)

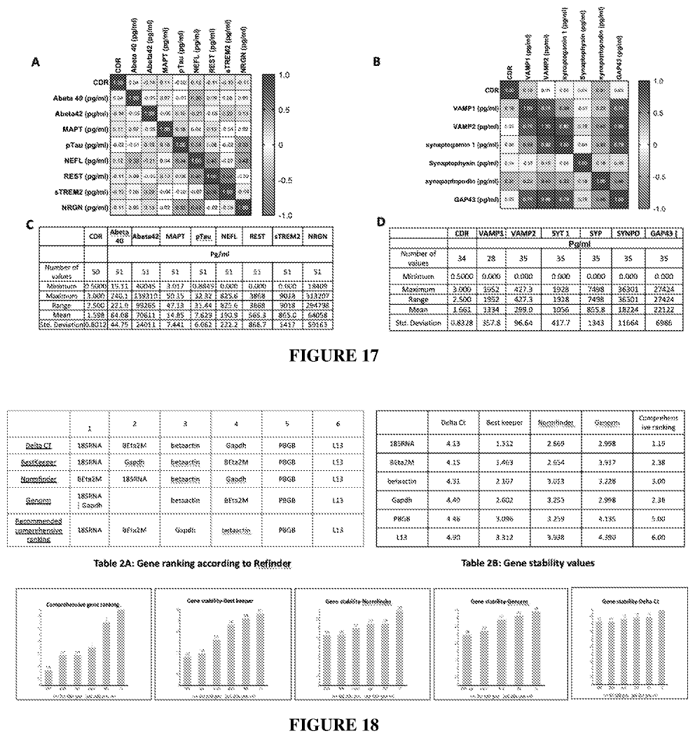

FIG. 17A shows the heat map of the Pearson correlation matrix between AD biomarkers and clinical dementia rating. A significant positive correlation was observed between NEFL and CDR score (r=0.12) with a mean value of 190.0a, positive correlation was observed between MAPT and CDR score (r=0.110 with a mean value of 14.85a, positive correlation was observed between Abeta40 and CDR score (r=0.04) with a mean value of 1.598. A significant negative correlation was observed between sTREM2 and CDR score (r=−0.15) with mean value 865, a negative correlation was observed between Abeta42 and CDR score (r=−0.14) with a mean value of 70611, a negative correlation was observed between REST and CDR score (r=−0.11) with a mean value of 565.3a, negative correlation was observed between NRGN and CDR score (r=−0.11) with a mean value of 6405, negative correlation was observed between pTau and CDR score (r=−0.02) with a mean value of 7.629. From the above results, it is evident that direct correlation was observed in MAPT and NEFL markers with CDR score, whereas reverse correlation was observed in Abet40, Abet42, pTau, NEFL, REST, sTREM2, and NRGN markers when compared with CDR score.

FIG. 17B shows the heat map of the Pearson correlation matrix between synaptic profile markers and clinical dementia rating score (CDR). A very significant positive correlation was observed between synaptopodin and CDR score (r=0.31) with a mean value of 18224, a significant positive correlation was observed between VAMP1 and CDR score (r=0.190 with a mean value of 1.661, a slight positive correlation was observed between VAMP2 and CDR score (r=0.19) with a mean value of 1334, A slight positive correlation was observed between synaptogamin1 and CDR score (r=0.04) with a mean value of 1056, a slight positive correlation was observed between synaptophysin and CDR (r=0.04) with a mean value of 855.8, a slight positive correlation was observed between GAP43 and CDR (r=0.03) with a mean value of 22122. From the above results, direct correlation was observed in all synaptic profile markers with CDR scores.

Youden index is a single statistic that captures the performance of a dichotomous diagnostic test. Informedness is its generalization to the multiclass case and estimates the probability of an informed decision. Youden's index is often used in conjunction with (ROC) analysis. The index is defined for all points of a ROC curve. The maximum value of the index may be used as a criterion for selecting the optimum cut-off point when a diagnostic test gives a numeric rather than a dichotomous result. The index is represented graphically as the height above the chance line, and it is also equivalent to the area under the curve subtended by a single operating point. YI is generated for the above protein parameters and is displayed in the table below

TABLE 5
Optimal cut Off score using youdens index

| | Optimal Cut | | | | | Youden's |
|---|---|---|---|---|---|---|
| | off value | Sensitivity % | 95% CI | Specificity % | 95% CI | index |
| Functional assay | | | | | | |
| Formaldehyde | 1139 | 39.58 | 27.02% to | 100 | 92.29% to | 138.58 |
| (ng/100 ul) | | | 53.69% | | 100.0% | |
| ACE (ng/mg) | 0.007215 | 87.5 | 75.30% to | 69.57 | 55.19% to | 156.07 |
| | | | 94.14% | | 80.92% | |
| Amyloid pathology | | | | | | |
| Abeta40 (pg/ml) | 232.1 | 95.83 | 86.02% to | 29.79 | 18.65% to | 124.62 |
| | | | 99.26% | | 43.98% | |
| Abeta42 (pg/ml) | 111.9 | 50 | 36.64% to | 87.23 | 74.83% to | 136.23 |
| | | | 63.36% | | 94.02% | |
| Tau pathology | | | | | | |
| Total tau (pg/ml) | 36.35 | 72 | 58.33% to | 69.57 | 55.19% to | 140.57 |
| | | | 82.53% | | 80.92% | |
| pTau (pg/ml) | 6.78 | 48.94 | 35.28% to | 95.56 | 85.17% to | 143.5 |
| | | | 62.76% | | 99.21% | |
| Axonal degeneration | | | | | | |
| NEFL | 452.9 | 35.29 | 23.63% to | 100 | 92.29% to | 134.29 |
| | | | 49.01% | | 100.0% | |
| Neuroinflammation | | | | | | |
| STREM2 | 0.7762 | 78.38 | 62.80% to | 82.61 | 62.86% to | 159.99 |
| | | | 88.61% | | 93.02% | |
| Neuroprotection | | | | | | |
| NRGN | 168.2 | 78.72 | 65.10% to | 82.61 | 69.28% to | 160.33 |
| | | | 88.01% | | 90.91% | |
| REST | 310.3 | 60 | 46.18% to | 82.61 | 69.28% to | 141.61 |
| | | | 72.39% | | 90.91% | |
| Synaptic profile | | | | | | |
| SYT1 | 1226 | 80 | 64.11% to | 92.5 | 80.14% to | 171.5 |
| | | | 89.96% | | 97.42% | |
| SYNPO | 25540 | 75.76 | 58.98% to | 80 | 65.24% to | 154.76 |
| | | | 87.17% | | 89.50% | |
| VAMP1 | 1866 | 96.77 | 83.81% to | 92.5 | 80.14% to | 188.27 |
| | | | 99.83% | | 97.42% | |
| VAMP2 | 621.1 | 100 | 89.28% to | 100 | 91.24% to | 199 |
| | | | 100.0% | | 100.0% | |
| GAP43 | 26552 | 96.88 | 84.26% to | 87.5 | 73.89% to | 183.38 |
| | | | 99.84% | | 94.54% | |
| SYP | 624.9 | 70.97 | 53.41% to | 89.74 | 76.42% to | 159.71 |
| | | | 83.90% | | 95.94% | |

Neuronal exosome RNA profiling—Exosome RNA is isolated from neuronal exosomes and characterized for their purity and quality using a bioanalyzer. Later a two-step PCR protocol is established for preamplification of RNA content in 1^{st} step and gene expression in the second step. Housekeeping genes are optimized by Refinder to identify the ideal housekeeping gene

### Optimization of Housekeeping Genes Using Refinder

ReFinder determined the gene expression stabilities of the 6 housekeeping genes from AD and control groups. Upon the input of Ct values, ReFinder invokes four commonly used computational programs, geNorm, NormFinder, BestKeeper, and comparative ΔCt method, to process those data, respectively. ReFinder aggregated the processed ranking results from each program to generate gene expression stability rank orders. Based on each program's rankings, ReFinder assigns an appropriate weight to each reference gene and then calculates the geometric mean of the weights for each gene to reach its overall final ranking, the comprehensive ranking order, among 6 housekeeping genes. We used the comprehensive rank order as our results.

The results of geNorm analysis showed that 18sRNA and Gapdh are high stability genes with 2.998 as stability value, followed by beta-actin, whereas PBGB and L13 are the least stable genes with 4.135 and 4.39 as stability values. The NormFinder algorithm demonstrated a different reference gene of choice. According to the stability value calculated by this software, Beta2M, with the least stability value of 2.654, ranked top as the best reference gene, followed by 18sRNA (FIG. 18, table 2A and 2B). Because the BestKeeper program is designed to assess the reference genes' reliability and determine a reliable normalization factor, but not to rank their reliability, we listed the candidate genes according to their standard deviation (SD) value. The BestKeeper program calculated 18sRNA and Gapdh as high stable reference genes with SD values of 1.312 and 1.463, whereas PBGB and L13 as least stable reference genes. The Delta ct program does not show much significant difference between the stability values of the genes. We have chosen 18sRNA, Gapdh, and Beta2M as the most stable reference gene based on the comprehensive ranking, which has a low stability value of 1.19 and 2.38.

### Molecular Diagnosis of AD Biomarkers—PCR Profiling

Table 6 shows the gene expression profiling of AD biomarkers in AD, early AD, other dementia, and control groups. Normalized delta ct values were used in gene expression analysis. Higher the delta ct value, low is the gene expression levels, lower the delta ct value, high is the gene expression levels.

TABLE 6
Descriptive statistics of genes at the RNA level. Amyloid markers (APP), tau
marker (MAPT), Immune checkpoints (PD1, PDL2), and Neurodegenerative marker
(NEFL) are significantly expressed in Alzheimer's patients compared to healthy
control. HC = Healthy control, AD = Alzheimer's disease

| | Gene | | | | |
|---|---|---|---|---|---|
| | HC | Early AD | AD | Others | HC vs AD |
| | Mean ± SD | Mean ± SD | Mean ± SD | Mean ± SD | p-value |
| APP | 4.470 (2.501) | 2.222 (5.091) | 1.830 (3.554) | 1.382 (4.170) | 0.0029** |
| MAPT | 3.705 (1.770) | 3.823 (5.053) | 2.487 (3.023) | 1.201 (3.960) | 0.0001*** |
| CTSD | 20.18 (30.65) | 2.747 (6.049) | 2.758 (3.162) | 1.902 (4.233) | 0.1997 |
| VSNL | 3.980 (2.151) | 1.323 (7.330) | 1.566 (3.684) | 0.3671 (4.571) | 0.0130* |
| PD1 | 1.301 (2.815) | −1.809 (2.642) | −1.810 (2.975) | −3.708 (2.952) | 0.0003*** |
| PDL2 | 1.497 (2.970) | 1.248 (1.574) | −1.898 (3.344) | −1.740 (2.154) | 0.0002*** |
| CTLA4 | −0.6952 (2.113) | 1.374 (1.057) | −1.412 (2.789) | −1.400 (2.189) | 0.8676 |
| PDL1 | 0.7352 (2.886) | 2.018 (1.325) | −0.6588 (3.714) | −1.639 (4.042) | 0.4786 |
| FABP | 2.697 (3.335) | 4.179 (0.7063) | 4.148 (2.947) | 3.162 (2.828) | 0.2011 |
| STMN2 | 0.8650 (2.941) | −0.4152 (0.8001) | −0.3287 (2.573) | −0.6611 (2.044) | 0.381 |
| COX2 | 0.3141 (2.471) | −0.1692 (1.705) | −0.6671 (2.490) | −0.3320 (2.092) | 0.3024 |
| NEFL | 4.291 (2.344) | 4.262 (0.7239) | 3.959 (2.630) | 3.184 (2.187) | 0.0201* |

### Amyloid and Tau Pathway

There is a significant increase (upregulated) in APP gene expression levels in AD (1.830±3.554, p=0.0029), early AD (2.222±5.091), and other dementia (1.382±4.170) groups when compared to healthy groups (Table 6, FIG. 19A). The ROC curve analysis showed significant AUC values of 0.7554, p<0.0001) (FIG. 19D). The optimal cut-off index was 2.834 with 62.5% sensitivity and 47.03% to 75.8% of 95% CI and 89.19% specificity and 75.29% to 95.71% of 95% CI with a likelihood ratio of 5.781 (Table 7). There is a significant increase (upregulated) in MAPT gene expression levels in AD (2.487±3.023, p=0.0001) and other dementia (1.201±3.960) when compared to the control group (Table 6, FIG. 19B). The ROC curve analysis showed significant AUC values of 0.8622, p<0.0001) (FIG. 19E). The optimal cut-off index was 2.818 with 90% sensitivity and 76.95% to 96.04% of 95% CI and 75.68% specificity and 59.98% to 86.64% of 95% CI with a likelihood ratio of 3.7 (Table 7).

TABLE 7
Optimal cut Off score using youdens index for Core biomarker's RNA expression

| | Cut off | | | | | Likelihood | Youdens |
|---|---|---|---|---|---|---|---|
| | value | Sensitivity % | 95% CI | Specificity % | 95% CI | ratio | index |
| APP | 2.834 | 62.5 | 47.03% to | 89.19 | 75.29% to | 5.781 | 150.69 |
| RNA | | | 75.78% | | 95.71% | | |
| MAPT | 2.818 | 90 | 76.95% to | 75.68 | 59.88% to | 3.7 | 164.68 |
| RNA | | | 96.04% | | 86.64% | | |
| NEFL | 4.585 | 85.71 | 72.16% to | 54.05 | 38.38% to | 1.866 | 138.76 |
| RNA | | | 93.28% | | 68.96% | | |

### Immune Check Point Markers

There is a high significant increase (upregulation) in PD1 gene expression levels in AD (−1.810±2.975, p=0.0003), early AD (−1.809±2.642,) and other dementia (−3.708±2.952) groups when compared to healthy groups (Table 6). There is a significant increase in PDL2 gene expression levels in AD (−1.898±3.344, p=0.0002) and other dementia (−1.740±2.154) compared to healthy groups. There is an increase in CTLA4 gene expression levels in AD (−1.412±2.789, p=0.8676) and other dementia (−1.400±2.189) compared with healthy groups. There is an increase in PDL1 gene expression levels in AD (−0.6588±3.714, p=0.4786) and other dementia groups (−1.639±4.042) compared with healthy groups (Table 6).

### Neuroinflammation Markers

There is an increase in STMN2 gene expression levels in AD (−0.3287±2.57, p=0.381), early AD (−0.4152±0.80,01), and other dementia (−0.6611±2.044) when compared with healthy groups. There is an increase in COX2 gene expression levels in AD (−0.6671±2.490. p=0.3024), early AD (−0.1692±1.,705), and other dementia (−0.3320±2.092) when compared with healthy control groups (Table 6).

### Axonal Degeneration Marker

There is a significant increase in NEFL gene expression levels in AD (3.959±2.630, p=0.0201) and other dementia (3.184±2.187) when compared with healthy groups (Table 6, FIG. 19C). The ROC curve analysis showed AUC values of 0.6892, p=0.0039 (FIG. 19F). The optimal cut-off index was 4.585 with 85.71% sensitivity and 72.16% to 93.28% of 95% CI and 54.05% specificity and 38.38% to 68.96% of 95% CI, a likelihood ratio of 1.86.

There is a decrease in FABP gene expression levels in AD (4.148±2.947, p=0.2011), early AD (4.179±0.7063) and, other dementia (3.162±2.828) when compared with healthy groups (Table 6).

### In vitro Risk Stratification of Alzheimer's Disease

The quantitative determination of one or more biomarkers for Alzheimer's disease can correlate the stage/progression of Alzheimer's disease with the expression levels. A correlation between the stage of Alzheimer's disease and the gene expression level can provide adequate means of prognosis and treatment. The correlation can provide a means for classifying a patient's risk status into a pre-determined risk category.

In one embodiment, the invention provides a method for the risk stratification of a subject for Alzheimer's disease, comprising determining the expression levels of at least one biomarker or combination of biomarkers having sufficient sensitivity and specificity in-vitro diagnosis/prognosis.

In one embodiment, one or more biomarkers used for in vitro risk stratification is selected from a group comprising Aβ40, Aβ42, TotalTau, p-tau181, NfL, sTrem2, Neurogranin, and REST.

In another embodiment, for risk stratification of a subject for Alzheimer's disease, a risk score is optionally calculated based on the relative threshold levels compared to healthy controls.

In yet another embodiment, the risk stratification of a subject for Alzheimer's disease on expression levels of one or more biomarkers/or the risk score.

### Assay Systems

In one aspect, the invention provides a system for diagnosis, prognosis, and/or risk stratification of Alzheimer's disease.

The system comprises one or more assays determining the expression of one or more biomarkers as provided herein, computer hardware, and software programs stored in computer-readable media extracting the expression level from the assays for the subject in the present invention.

In one embodiment, the system comprises a biomarker or genetic marker expression evaluation element configured for evaluating the expression level of at least one gene/protein in a sample from a subject to obtain a gene expression result, wherein said at least one gene is selected from Table 1.

In one embodiment, the invention provides an assay system for determining Alzheimer's disease risk in a subject, said system comprising:
a. a plurality of antibodies specific to a biomarker panel as disclosed herein, wherein the antibodies are selected from a group comprising anti-Abeta 40 antibodies, anti-Abeta 42 antibodies, anti-Total Tau antibodies, anti-Phospho 181 Tau antibodies, anti-sTREM2 antibodies, anti-NEFL antibodies, anti-Neurogranin antibodies, anti-REST antibodies optionally, along with antibodies selected from a group comprising anti-Synaptogamin-1 antibodies, anti-Synaptopodin antibodies, anti-Synaptophysin antibodies, anti-Vamp-2 antibodies, anti-VAMP-1 antibodies, anti-GAP 43 antibodies, anti-CTSD antibodies, anti-VSNL antibodies, anti-PD1 antibodies, anti-PDL2 antibodies, anti-CTLA4 antibodies, anti-PDL1 antibodies, anti-FABP antibodies, anti-NEFH antibodies, anti-COX2 antibodies and anti-PGF antibodies; and
b. means to measure the expression levels of the antibodies of step (a) through immunoassay systems; and
c. a processing unit configured to estimate the risk of Alzheimer's disease.

In another embodiment, the invention provides an assay system for determining Alzheimer's disease risk in a subject, said system comprising:
a. a plurality of primer set for amplifying a biomarker panel as disclosed herein, wherein primer sets are selected from a group comprising:
   i. SEQ ID NO: 1 and SEQ ID NO: 2, wherein the primer set is specific to APP gene;
   ii. SEQ ID NO: 3 and SEQ ID NO: 4, wherein the primer set is specific to MAPT gene;
   iii. SEQ ID NO: 5 and SEQ ID NO: 6, wherein the primer set is specific to the CTSD gene;
   iv. SEQ ID NO: 7 and SEQ ID NO: 8, wherein the primer set is specific to the VSNL gene;
   v. SEQ ID NO: 9; SEQ ID NO: 10, wherein the primer set is specific to the PD1 gene.
   vi. SEQ ID NO: 11; SEQ ID NO: 12, wherein the primer set is specific to the PDL2 gene.
   vii. SEQ ID NO: 13, SEQ ID NO: 14, wherein the primer set is specific to the CTLA4 gene;
   viii. SEQ ID NO: 15; SEQ ID NO: 16, wherein the primer set is specific to the PDL1 gene;
   ix. SEQ ID NO: 17; SEQ ID NO: 18, wherein the primer set is specific to the FABP gene;
   x. SEQ ID NO: 19, SEQ ID NO: 20, wherein the primer set is specific to NEFH gene;
   xi. SEQ ID NO: 21, SEQ ID NO: 22, wherein the primer set is specific to the sTREM2 gene;
   xii. SEQ ID NO: 23; SEQ ID NO: 24, wherein the primer set is specific to the COX2 gene;
   xiii. SEQ ID NO: 25; SEQ ID NO: 26, wherein the primer set is specific to the NEFL gene;

xiv. SEQ ID NO: 27 and SEQ ID NO: 28, wherein the primer set is specific to the PGF gene, and
b. means to measure the expression levels of the antibodies of step (a) through PCR-based assay systems; and
c. a processing unit configured to estimate the risk of Alzheimer's disease.

In another embodiment, the biomarker or genetic marker expression evaluation element comprises at least one reagent to detect and quantify at least one biomarker described herein.

The system comprises a risk score determination element in another embodiment, wherein the risk score determination element employs said gene expression result.

In another embodiment, the risk score can be used for prognosis, diagnosis and/or risk stratification of a subject for Alzheimer's disease.

In one embodiment, an assay system 100 comprises a processing unit 102, a memory 104, an I/O interface 106, and a display unit 108. The processing unit 102 is configured to receive via the I/O interface 106 the measured expression levels of the biomarker panel and the risk associated with the biomarker panel's measured expression levels. In one embodiment, the processing unit 102 receives the expression levels of the biomarker panel measured by a measuring unit (not shown) of the assay system 100 from a blood sample of a subject. It determines Alzheimer's disease risk associated with the subject.

In one embodiment, the processing unit 102 receives measured expression levels of biomarkers and determines risk score associated with the received measured expression levels of biomarkers. In one example, the processing unit 102 compares the received measured expression levels of the biomarker panel with standard expression levels to determine the risk score. For example, the standard expression levels may be derived historically from many subjects or may refer to universally accepted expression levels stored in the memory 104. Upon determining the risk score, the processing unit 102 estimates one of low risk, medium risk, and high risk of Alzheimer's disease and display via the display unit 108. For example, processing unit 102 estimates the low risk if the risk score relates to less likelihood of Alzheimer's disease. In another example, processing unit 102 calculates the medium risk if the risk score relates to average likelihood of Alzheimer's disease. In yet another example, processing unit 102 estimates the high risk if the risk score relates to increased likelihood of Alzheimer's disease.

In another embodiment, the assay system 100 may be integrated with a software application capable of assessing the Alzheimer's disease risk based on expression levels of biomarkers measured by the assay system. In yet another embodiment, the processing unit 102 may be configured to predict the Alzheimer's disease risk based on expression levels of biomarkers using an Artificial Intelligent (AI) engine (not shown in FIG. 1). The AI engine can be trained with plurality of expression levels of biomarkers recorded over a period of time and associated Alzheimer's disease risk level that were previously assessed. Based on training, the AI engine predicts the Alzheimer's disease risk level of a subject in real-time based on current expression levels of biomarkers. Thus, the assay system 100 is capable of assessing the Alzheimer's disease risk level of a subject with improved accuracy and in less time.

In another embodiment, the invention provides an assay system comprising a processing unit is configured to:
a. receive the measured expression levels of biomarker panel as disclosed herein;
b. determine a risk score associated with the measured expression levels by comparing the received measured expression levels of biomarker panel with standard expression levels; and
c. estimate the risk of Alzheimer's disease of a subject based on the risk score thus determined.

A computer-implemented method for determining the risk of an individual for having Alzheimer's disease risk said method comprising:
a. receiving, by a processing unit of an assay system as described herein, expression levels of biomarkers selected from a group comprising Abeta 40, Abeta 42, Total Tau, Phospho 181 Tau, sTREM2, NEFL, Neurogranin, and REST, optionally along with at least one biomarker selected from a group comprising Synaptogamin-1, Synaptopodin, Synaptophysin, Vamp-2, VAMP-1, GAP 43, CTSD, VSNL, PD1, PDL2, CTLA4, PDL1, FABP, NEFH, COX2, and PGF, wherein the expression levels are measured using the assay system;
b. determining, by the processing unit, a risk score associated with the received expression levels of biomarkers by comparing the received measured expression levels of biomarker panel with standard expression levels; and
c. estimating, by the processing unit, the risk level of Alzheimer's disease based on the risk score thus determined.

### Kits

In one embodiment, the invention provides a kit.

In another embodiment, the kit comprises reagents to detect and quantify the set of biomarkers described herein and instruction material for using the kit.

In another embodiment, one or more biomarkers listed in Table 1 can be quantified using the kit.

In one embodiment, the kit is useful for the diagnosis or prognosis of Alzheimer's disease.

In another embodiment, the kit is useful for pre-symptomatic diagnosis of Alzheimer's disease.

In yet another embodiment, the kit is useful for risk assessment or risk stratification for conducting clinical trials of Alzheimer's disease drugs.

In yet another embodiment, the kit is useful for evaluating the prognosis of treatments for Alzheimer's disease.

In one embodiment, the invention provides a kit for determining Alzheimer's disease risk in a subject comprising anti-Abeta 40 antibodies, anti-Abeta 42 antibodies, anti-Total Tau antibodies, anti-Phospho 181 Tau antibodies, anti-sTREM2 antibodies, anti-NEFL antibodies, anti-Neurogranin antibodies, anti-REST antibodies optionally, along with antibodies selected from a group comprising anti-Synaptogamin-1 antibodies, anti-Synaptopodin antibodies, anti-Synaptophysin antibodies, anti-Vamp-2 antibodies, anti-VAMP-1 antibodies, anti-GAP 43 antibodies, anti-CTSD antibodies, anti-VSNL antibodies, anti-PD1 antibodies, anti-PDL2 antibodies, anti-CTLA4 antibodies, anti-PDL1 antibodies, anti-FABP antibodies, anti-NEFH antibodies, anti-COX2 antibodies, and anti-PGF antibodies, and optionally a primer set capable of amplifying biomarkers selected from a biomarker panel as described herein.

In another embodiment, the invention provides a kit that comprises at least one reagent selected from buffer solutions, protein blocking agents, primary antibodies, and secondary antibodies.

In yet another embodiment, the invention provides a comprising one or more primers selected from the following primer sets:
a. SEQ ID NO: 1 and SEQ ID NO: 2, wherein the primer set is specific to APP gene;
b. SEQ ID NO: 3 and SEQ ID NO: 4, wherein the primer set is specific to the MAPT gene;
c. SEQ ID NO: 5 and SEQ ID NO: 6, wherein the primer set is specific to the CTSD gene;
d. SEQ ID NO: 7 and SEQ ID NO: 8, wherein the primer set is specific to the VSNL gene;
e. SEQ ID NO: 9 and SEQ ID NO: 10, wherein the primer set is specific to the PD1 gene;
f. SEQ ID NO: 11 and SEQ ID NO: 12, wherein the primer set is specific to the PDL2 gene;
g. SEQ ID NO: 13 and SEQ ID NO: 14, wherein the primer set is specific to the CTLA4 gene;
h. SEQ ID NO: 15 and SEQ ID NO: 16, wherein the primer set is specificthe to the PDL1 gene;
i. SEQ ID NO: 17 and SEQ ID NO: 18, wherein the primer set is specific to the FABP gene;
j. SEQ ID NO: 19 and SEQ ID NO: 20, wherein the primer set is specific to the NEFH gene.
k. SEQ ID NO: 21, SEQ ID NO: 22, wherein the primer set is specific to the STREM2 gene;
l. SEQ ID NO: 23 and SEQ ID NO: 24, wherein the primer set is specific to the COX2 gene;
m. SEQ ID NO: 25 and SEQ ID NO: 26, wherein the primer set is specific to the NEFL gene; and
n. SEQ ID NO: 27 and SEQ ID NO: 28, wherein the primer set is specific to the PGF gene.

### In vitro Methods of Measurement and Diagnosis of AD

In another embodiment, the invention provides a method of measuring expression of biomarkers for assessing Alzheimer's disease risk, comprising the steps of
a. obtaining a biological sample from a subject having a risk of Alzheimer's disease.
b. measuring the expression levels of biomarkers of the biomarker panel as described herein using an assay system as described herein.
   In another embodiment, the invention provides an in vitro method of diagnosing Alzheimer's disease risk in a subject comprising:
a. obtaining a biological sample from a subject having a risk of Alzheimer's disease;
b. measuring the expression levels of biomarkers of the biomarker panel as described herein;
c. determining a risk score associated with the expression levels of biomarkers by comparing the measured expression levels of biomarker panel with standard expression levels; and
d. estimating the risk level of Alzheimer's disease based on the risk score thus determined.
   In another embodiment, the invention provides an in vitro method of diagnosing Alzheimer's disease risk,
wherein the measurement is done using an immunoassay system or a PCR-based assay system.
   In another embodiment, the invention provides an in vitro method of diagnosing Alzheimer's disease risk,
wherein the measurement is done using a kit as described herein.

### Treatment of Alzheimer's Disease

The appropriate therapeutic intervention for the treatment of Alzheimer's disease is the general administration of one or more therapeutic agents/compositions for treatment or alleviation of symptoms associated with Alzheimer's disease.

The present invention would aid as a companion diagnostic for real-time monitoring of the progression of the treatment. Based on the progression, the clinical practitioner may make appropriate changes in the therapeutic modalities.

### Advantages of the Invention

The invention provides inexpensive, rapid, minimal-invasive, and objective diagnostic and/or prognostic determination of Alzheimer's disease and can be extended to different AD stages. Non-requirement of highly trained personnel for sample collection provides a huge advantage and can lead to real-time disease monitoring.

Further, the early diagnosis of Alzheimer's disease is possible with this invention which can potentially save a number of lives and may alter the course of AD trajectory. The invention can also be used for real-time assessment of the outcome of clinical trials.

The quantification of expression levels of one or more biomarkers listed in Table 1 provides a sound basis for reaching a conclusive determination regarding the disease's extent. Further, the development of a risk score would help standardize the therapeutic modalities for the treatment of Alzheimer's disease. Companion diagnostics can be prepared for individual pathways (amyloid, tau and neurodegeneration pathology, axonal and synaptic degeneration, neuroinflammation, glial activation, alpha-synuclein pathology, and others.

1. (canceled)

2. An assay system for determining Alzheimer's disease risk in a subject, said system comprising:
a) a plurality of antibodies selected from a group comprising anti-Abeta 40 antibodies, anti-Abeta 42 antibodies, anti-Total Tau antibodies, anti-Phospho 181 Tau antibodies, anti-sTREM2 antibodies, anti-NEFL antibodies, anti-Neurogranin antibodies, anti-REST antibodies optionally, along with antibodies selected from a group comprising anti-Synaptogamin-1 antibodies, anti-Synaptopodin antibodies, anti-Synaptophysin antibodies, anti-Vamp-2 antibodies, anti-VAMP-1 antibodies, anti-GAP 43 antibodies, anti-CTSD antibodies, anti-VSNL antibodies, anti-PD1 antibodies, anti-PDL2 antibodies, anti-CTLA4 antibodies, anti-PDL1 antibodies, anti-FABP antibodies, anti-NEFH antibodies, anti-COX2 antibodies and anti-PGF antibodies; and
b) means to measure the expression levels of the antibodies of step (a) through immunoassay systems; and
c) a processing unit configured to estimate the risk of Alzheimer's disease.

3. The assay system of claim 2, said system comprising:
a) a plurality of primer set selected from a group comprising:
i. SEQ ID NO: 1 and SEQ ID NO: 2, wherein the primer set is specific to APP gene;
ii. SEQ ID NO: 3 and SEQ ID NO: 4, wherein the primer set is specific to MAPT gene;
iii. SEQ ID NO: 5 and SEQ ID NO: 6, wherein the primer set is specific to CTSD gene;
iv. SEQ ID NO: 7 and SEQ ID NO: 8, wherein the primer set is specific to VSNL gene;
v. SEQ ID NO: 9; SEQ ID NO: 10, wherein the primer set is specific to PD1 gene;
vi. SEQ ID NO: 11; SEQ ID NO: 12, wherein the primer set is specific to PDL2 gene;
vii. SEQ ID NO: 13, SEQ ID NO: 14, wherein the primer set is specific to CTLA4 gene;
viii. SEQ ID NO: 15; SEQ ID NO: 16, wherein the primer set is specific to PDL1 gene;
ix. SEQ ID NO: 17; SEQ ID NO: 18, wherein the primer set is specific to FABP gene; SEQ ID NO: 19, SEQ ID NO: 20, wherein the primer set is specific to NEFH gene;
x. SEQ ID NO: 21, SEQ ID NO: 22, wherein the primer set is specific to STREM2 gene;
xi. SEQ ID NO: 23; SEQ ID NO: 24, wherein the primer set is specific to COX2 gene;
xii. SEQ ID NO: 25; SEQ ID NO: 26, wherein the primer set is specific to NEFL gene;
xiii. SEQ ID NO: 27 and SEQ ID NO: 28, wherein the primer set is specific to PGF gene, and
b) means to measure the expression levels of the gene expression of step (a) through PCR-based assay systems; and
c) a processing unit configured to estimate the risk of Alzheimer's disease.

4. The assay system of claim 2, wherein the processing unit is configured to:
a) receive the measured expression levels of biomarker panel as claimed in claim 1;
b) determine a risk score associated with the measured expression levels by comparing the received measured expression levels of biomarker panel with standard expression levels; and
c) estimate the risk of Alzheimer's disease of a subject based on the risk score thus determined.

5. A kit for determining Alzheimer's disease risk in a subject comprising anti-Abeta 40 antibodies, anti-Abeta 42 antibodies, anti-Total Tau antibodies, anti-Phospho 181 Tau antibodies, anti-sTREM2 antibodies, anti-NEFL antibodies, anti-Neurogranin antibodies, anti-REST antibodies optionally, along with antibodies selected from a group comprising anti-Synaptogamin-1 antibodies, anti-Synaptopodin antibodies, anti-Synaptophysin antibodies, anti-Vamp-2 antibodies, anti-VAMP-1 antibodies, anti-GAP 43 antibodies, anti-CTSD antibodies, anti-VSNL antibodies, anti-PD1 antibodies, anti-PDL2 antibodies, anti-CTLA4 antibodies, anti-PDL1 antibodies, anti-FABP antibodies, anti-NEFH antibodies, anti-COX2 antibodies and anti-PGF antibodies.

6. The kit as claimed in claim 5, further comprising at least one reagent selected from buffer solutions, protein blocking agents, primary antibodies, and secondary antibodies.

7. The kit as claimed in claim 5, further comprising one or more primers selected from the following primer sets:
a. SEQ ID NO: 1 and SEQ ID NO: 2, wherein the primer set is specific to APP gene;
b. SEQ ID NO: 3 and SEQ ID NO: 4, wherein the primer set is specific to MAPT genes;
c. SEQ ID NO: 5 and SEQ ID NO: 6, wherein the primer set is specific to CTSD gene;
d. SEQ ID NO: 7 and SEQ ID NO: 8, wherein the primer set is specific to VSNL gene;
e. SEQ ID NO: 9 and SEQ ID NO: 10, wherein the primer set is specific to PD1 gene;
f. SEQ ID NO: 11 and SEQ ID NO: 12, wherein the primer set is specific to PDL2 gene;
g. SEQ ID NO: 13 and SEQ ID NO: 14, wherein the primer set is specific to CTLA4 gene;
h. SEQ ID NO: 15 and SEQ ID NO: 16, wherein the primer set is specific to PDL1 gene;
i. SEQ ID NO: 17 and SEQ ID NO: 18, wherein the primer set is specific to FABP gene;
j. SEQ ID NO: 19 and SEQ ID NO: 20, wherein the primer set is specific to NEFH gene;
k. SEQ ID NO: 21, SEQ ID NO: 22, wherein the primer set is specific to STREM2 gene;
l. SEQ ID NO: 23 and SEQ ID NO: 24, wherein the primer set is specific to COX2 gene;
m. SEQ ID NO: 25 and SEQ ID NO: 26, wherein the primer set is specific to NEFL gene; and
n. SEQ ID NO: 27 and SEQ ID NO: 28, wherein the primer set is specific to PGF gene.

8. (canceled)

9. (canceled)

10. An in vitro method of diagnosing Alzheimer's disease risk in a subject comprising:
a. obtaining a biological sample from a subject having a risk of Alzheimer's disease;
b. measuring the expression levels of one or more biomarkers selected from Abeta 40, Abeta 42, Total Tau, Phospho 181 Tau, sTREM2, NEFL, Neurogranin and REST, optionally along with at least one biomarker selected from a group comprising Synaptogamin-1, Synaptopodin, Synaptophysin, Vamp-2, VAMP-1, GAP 43, CTSD, VSNL, PD1, PDL2, CTLA4, PDL1, FABP, NEFH, COX2 and PGF;
c. determining a risk score associated with the expression levels of biomarkers by comparing the measured expression levels of biomarker panel with standard expression levels; and
d. estimating the risk level of Alzheimer's disease based on the risk score thus determined.

11. (canceled)

12. (canceled)


SEQUENCE LISTING (40 sequences)

SEQ ID NO: 1 - DNA, 21 bp, Artificial Sequence
  Forward primer for amplifying APP gene
ccatcagggaccaaaacctgc

SEQ ID NO: 2 - DNA, 23 bp, Artificial Sequence
  Reverse Primer for amplifying APP gene
cagttcagggtagacttcttggc

SEQ ID NO: 3 - DNA, 20 bp, Artificial Sequence
  Forward primer for amplifying MAPT gene
ccagctctgcgactaagcaa

SEQ ID NO: 4 - DNA, 21 bp, Artificial Sequence
  Reverse Primer for amplifying MAPT gene
gcgatcccctgattttggagg

SEQ ID NO: 5 - DNA, 20 bp, Artificial Sequence
  Forward primer for amplifying CTSD gene
acaggcacttccctcatggt

SEQ ID NO: 6 - DNA, 20 bp, Artificial Sequence
  Reverse Primer for amplifying CTSD gene
ggggatcatgtactcgccct

SEQ ID NO: 7 - DNA, 20 bp, Artificial Sequence
  Forward primer for amplifying VSNL gene
cggcttaagcgtttacccga

SEQ ID NO: 8 - DNA, 20 bp, Artificial Sequence
  Reverse Primer for amplifying VSNL gene
gcttccccatcctgcagtta

SEQ ID NO: 9 - DNA, 20 bp, Artificial Sequence
  Forward primer for amplifying PD1 gene
cgtggcctatccactcctca

SEQ ID NO: 10 - DNA, 20 bp, Artificial Sequence
  Reverse Primer for amplifying PD1 gene
atcccttgtcccagccactc

SEQ ID NO: 11 - DNA, 22 bp, Artificial Sequence
  Forward primer for amplifying PDL2 gene
cagtgctatctgaacctgtggt

SEQ ID NO: 12 - DNA, 20 bp, Artificial Sequence
  Reverse Primer for amplifying PDL2 gene
gccaggtgttggctagtctt

SEQ ID NO: 13 - DNA, 22 bp, Artificial Sequence
  Forward primer for amplifying CTLA4 gene
catgatggggaatgagttgacc

SEQ ID NO: 14 - DNA, 23 bp, Artificial Sequence
  Reverse Primer for amplifying CTLA4 gene
tcagtccttggatagtgaggttc

SEQ ID NO: 15 - DNA, 20 bp, Artificial Sequence
  Forward primer for amplifying PDL1 gene
aaatggaacctggcgaaagc

SEQ ID NO: 16 - DNA, 20 bp, Artificial Sequence
  Reverse Primer for amplifying PDL1 gene
gatgagcccctcaggcattt

SEQ ID NO: 17 - DNA, 20 bp, Artificial Sequence
  Forward primer for amplifying FABP gene
tgggcacctggaagctagtg

SEQ ID NO: 18 - DNA, 19 bp, Artificial Sequence
  Reverse Primer for amplifying FABP gene
agcaaaacccacaccgagt

SEQ ID NO: 19 - DNA, 21 bp, Artificial Sequence
  Forward primer for amplifying NEFH gene
cgaggagtggttccgagtgag

SEQ ID NO: 20 - DNA, 20 bp, Artificial Sequence
  Reverse Primer for amplifying NEFH gene
gcgcatagcgtctgtgttca

SEQ ID NO: 21 - DNA, 20 bp, Artificial Sequence
  Forward primer for amplifying STMN2 gene
ttcagcaagatggcggagga

SEQ ID NO: 22 - DNA, 22 bp, Artificial Sequence
  Reverse Primer for amplifying STMN2 gene
gcctctcattgcttctctcctt

SEQ ID NO: 23 - DNA, 23 bp, Artificial Sequence
  Forward primer for amplifying COX2 gene
ggagaaaactgctcaacaccgga

SEQ ID NO: 24 - DNA, 21 bp, Artificial Sequence
  Reverse Primer for amplifying COX2 gene
gtgcactgtgtttggagtggg

SEQ ID NO: 25 - DNA, 22 bp, Artificial Sequence
  Forward primer for amplifying NEFL gene
gctatgcaggacacgatcaaca

SEQ ID NO: 26 - DNA, 21 bp, Artificial Sequence
  Reverse Primer for amplifying NEFL gene
tcacgttgaggaggtcttggt

SEQ ID NO: 27 - DNA, 22 bp, Artificial Sequence
  Forward primer for amplifying PGF gene
ctcgtcagaggtggaagtggta

SEQ ID NO: 28 - DNA, 20 bp, Artificial Sequence
  Reverse Primer for amplifying PGF gene
cagggagacacaggatgggc

SEQ ID NO: 29 - DNA, 20 bp, Artificial Sequence
  Forward primer for amplifying L13 gene
tcctttccgctcggctgttt

SEQ ID NO: 30 - DNA, 19 bp, Artificial Sequence
  Reverse Primer for amplifying L13 gene
gggccttacgtctgcggat

SEQ ID NO: 31 - DNA, 21 bp, Artificial Sequence
  Forward primer for amplifying 18s gene
ctgagaaacggctaccacatc

SEQ ID NO: 32 - DNA, 22 bp, Artificial Sequence
  Reverse Primer for amplifying 18s gene
gcctcgaaagagtcctgtattg

SEQ ID NO: 33 - DNA, 23 bp, Artificial Sequence
  Forward primer for amplifying Bactin gene
gtctgccttggtagtggataatg

SEQ ID NO: 34 - DNA, 20 bp, Artificial Sequence
  Reverse Primer for amplifying Bactin gene
tcgaggacgccctatcatgg

SEQ ID NO: 35 - DNA, 21 bp, Artificial Sequence
  Forward primer for amplifying GAPDH gene
ccactaggcgctcactgttct

SEQ ID NO: 36 - DNA, 22 bp, Artificial Sequence
  Reverse Primer for amplifying GAPDH gene
gaccaaatccgttgactccgac

SEQ ID NO: 37 - DNA, 21 bp, Artificial Sequence
  Forward primer for amplifying B2M gene
cctgccgtgtgaaccatgtga

SEQ ID NO: 38 - DNA, 23 bp, Artificial Sequence
  Reverse Primer for amplifying B2M gene
gctgcttacatgtctcgatccca

SEQ ID NO: 39 - DNA, 20 bp, Artificial Sequence
  Forward primer for amplifying PBGB gene
cagcctactttccaagcgga

SEQ ID NO: 40 - DNA, 22 bp, Artificial Sequence
  Reverse Primer for amplifying PBGB gene
cctgtggtggacatagcaatga